SEQUENCE LISTING (26 sequences)

SEQ ID NO: 1 - protein, 8 aa, synthetic construct
  REGION 1..8: note = CDR1-H
GYTFTSYN

SEQ ID NO: 2 - protein, 8 aa, synthetic construct
  REGION 1..8: note = CDR2-H
IYPGNGDT

SEQ ID NO: 3 - protein, 9 aa, synthetic construct
  REGION 1..9: note = CDR3-H
ARGGTGFAY

SEQ ID NO: 4 - protein, 6 aa, synthetic construct
  REGION 1..6: note = CDR1-L
QSVSND

SEQ ID NO: 5 - protein, 9 aa, synthetic construct
  REGION 1..9: note = CDR3-L
QQDYSSPWT

SEQ ID NO: 6 - protein, 440 aa, synthetic construct
  REGION 1..440: note = Full aa sequence of 4C11 (VH + mouse IgG1 CH)
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARGGTGFAYWGQGTLVTVSAAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 7 - protein, 214 aa, synthetic construct
  REGION 1..214: note = Full aa sequence of 4C11 (VL + mouse Kappa CL)
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 8 - protein, 101 aa, synthetic construct
  REGION 1..101: note = VL aa sequence of humanized variant of 4C11
EIVMTQSPATLSVSPGERATLSCKASQSVSNDVAWYQQKPGKAPKLLIYYASNRYTGIPPRFSGSGYGTDFTLTINNIESEDAAYYFCQQDYSSPWTFGQG

SEQ ID NO: 9 - protein, 101 aa, synthetic construct
  REGION 1..101: note = VL aa sequence of humanized variant of 4C11
DIQMTQSPSSLSASVGDRVTITCKASQSVSNDVAWFQQRPGQSPRRLIYYASNRYTGVPSRFSGSGSGTDFTFTISSLEAEDAATYYCQQDYSSPWTFGQG

SEQ ID NO: 10 - protein, 101 aa, synthetic construct
  REGION 1..101: note = VL aa sequence of humanized variant of 4C11
DIQMTQSPSSLSASVGDRVTITCKASQSVSNDVAWYQQKPGQAPRLLIYYASNRYTGIPPRFSGSGYGTDFTLTINNIESEDAAYYFCQQDYSSPWTFGQG

SEQ ID NO: 11 - protein, 101 aa, synthetic construct
  REGION 1..101: note = VL aa sequence of humanized variant of 4C11
DIQMTQSPSSLSASVGDRVTITCKASQSVSNDVAWYLQKPGQSPQLLIYYASNRYTGVPSRFSGSGSGTDFTFTISSLEAEDAATYYCQQDYSSPWTFGQG

SEQ ID NO: 12 - protein, 101 aa, synthetic construct
  REGION 1..101: note = VL aa sequence of humanized variant of 4C11
DIVMTQTPLSLPVTPGEPASISCKASQSVSNDVAWYQQKPGQAPRLLIYYASNRYTGIPPRFSGSGYGTDFTLTINNIESEDAAYYFCQQDYSSPWTFGQG

SEQ ID NO: 13 - protein, 101 aa, synthetic construct
  REGION 1..101: note = VL aa sequence of humanized variant of 4C11
EIVMTQSPATLSVSPGERATLSCRASQSVSNDVAWYQQKPGQAPRLLIYYASNRYTGIPARFSGSGSGTEFTLTISSLQSEDFAVYYCQQDYSSPWTFGQG

SEQ ID NO: 14 - protein, 109 aa, synthetic construct
  REGION 1..109: note = VH aa sequence of humanized variant of 4C11
QVQLVQSGAEVKKPGASVKVSCKASGYTFTSYNMHWVRQATGQGLEWMGAIYPGNGDTSYNQKFKGRVTITADKSTSTAYMELSSLRSEDTAVYYCARGGTGFAYWGQG

SEQ ID NO: 15 - protein, 109 aa, synthetic construct
  REGION 1..109: note = VH aa sequence of humanized variant of 4C11
QVQLQQSGPGLVKPSQTLSLTCAISGYTFTSYNMHWIRQPPGKGLEWIGAIYPGNGDTSYNQKFKGRVTITADKSTSTAYMELSSLRSEDTAVYYCARGGTGFAYWGQG

SEQ ID NO: 16 - protein, 109 aa, synthetic construct
  REGION 1..109: note = VH aa sequence of humanized variant of 4C11
QVQLQQSGPGLVKPSQTLSLTCAISGYTFTSYNMHWVRQATGQGLEWMGAIYPGNGDTSYNQKFKGRLTISKDTSKNQVVLTMTNMDPVDTATYYCARGGTGFAYWGQG

SEQ ID NO: 17 - protein, 109 aa, synthetic construct
  REGION 1..109: note = VH aa sequence of humanized variant of 4C11
EVQLVQSGAEVKKPGESLRISCKGSGYTFTSYNMHWVRQATGQGLEWMGAIYPGNGDTSYNQKFKGRFTISRDDSKNTAYLQMNSLKTEDTAVYYCARGGTGFAYWGQG

SEQ ID NO: 18 - protein, 109 aa, synthetic construct
  REGION 1..109: note = VH aa sequence of humanized variant of 4C11
QVQLQESGPGLVKPSQTLSLTCTVSGYTFTSYNMHWVRQAPGQGLEWMGAIYPGNGDTSYNQKFKGRVTISVDTSKNQFSLKLSSVTAADTAVYYCARGGTGFAYWGQG

SEQ ID NO: 19 - protein, 109 aa, synthetic construct; identical to SEQ ID NO: 14
  REGION 1..109: note = VH aa sequence of humanized variant of 4C11

SEQ ID NO: 20 - protein, 109 aa, synthetic construct
  REGION 1..109: note = VH aa sequence of humanized variant of 4C11
QVQLQQSGPGLVKPSQTLSLTCAISGYTFTSYNMHWVRQATGQGLEWMGAIYPGNGDTSYNQKFKGRVTITADKSTSTAYMELSSLRSEDTAVYYCARGGTGFAYWGQG

SEQ ID NO: 21 - protein, 108 aa, synthetic construct
  REGION 1..108: note = VH aa sequence of humanized variant of 4C11
QVQLVQSGAEVKKPGASVKVSCKASGYTFTSYNMHWVRQAPGQGLEWMGAIYPGNGDTSYAQKFKGRVTMTRDTSTSTVYMELSSLRSEDTAVYYCARGGTGFAYWGQ

SEQ ID NO: 22 - protein, 5 aa, synthetic construct
  REGION 1..5: note = aa seq. of CDR1-H (Kabat)
SYNMH

SEQ ID NO: 23 - protein, 17 aa, synthetic construct
  REGION 1..17: note = aa seq. of CDR2-H (Kabat)
AIYPGNGDTSYNQKFKG

SEQ ID NO: 24 - protein, 7 aa, synthetic construct
  REGION 1..7: note = aa seq. of CDR3-H (Kabat)
GGTGFAY

SEQ ID NO: 25 - protein, 11 aa, synthetic construct
  REGION 1..11: note = aa seq. of CDR1-L (Kabat)
KASQSVSNDVA

SEQ ID NO: 26 - protein, 7 aa, synthetic construct
  REGION 1..7: note = aa seq. of CDR2-L (Kabat)
YASNRYT

(19) United States
(12) Patent Application Publication
(10) Pub. No.: US 2024/0342322 A1
(43) Pub. Date: Oct. 17, 2024

(54) Netrin-1 detection, companion test and therapy based on radiations

(71) Applicants: NETRIS PHARMA, LYON (FR); Centre national de la recherche scientifique, PARIS (FR); INSTITUT NATIONAL DE LA SANTE ET DE LA RECHERCHE MEDICAL (INSERM), PARIS (FR); Université Claude Bernard Lyon 1, VILLEURBANNE (FR); CENTRE LEON BERARD, LYON (FR); HOSPICES CIVILS DE LYON, LYON (FR)

(72) Inventors: Mathieu RICHAUD, LYON (FR); Jennifer WISCHHUSEN, DOSSENHEIM (DE); David NEVES, VERNAISON (FR); Patrick MEHLEN, SEREZIN-DE-LA-TOUR (FR); David SARRUT, LA TOUR DU PIN (FR); Benjamin GIBERT, VILLEURBANNE (FR); David KRYZA, SAINT DIDIER AU MONT D'OR (FR)

(21) Appl. No.: 18/291,972

(22) PCT Filed: Jul. 26, 2022
(86) PCT No.: PCT/EP2022/070944

§ 371 Date: Jan. 25, 2024

(30) Foreign Application Priority Data
Jul. 27, 2021 (EP) 21306040.3

(51) Int. Cl. A61K 51/10; A61P 35/00; C07K 16/22

(52) U.S. Cl. CPC A61K 51/1021; A61K 51/1096; A61P 35/00; C07K 16/22; A61K 2121/00; A61K 2123/00; C07K 2317/54; C07K 2317/55; C07K 2317/92

(57) ABSTRACT

The present invention is based on the finding that Netrin-(1) is retained in a stickier manner in the cell matrix at the cell periphery of the cancer cells, whereas Netrin-(1) is expressed in adults specifically in some tumors. It is also shown herein that Netrin-(1) is expressed very early during tumor formation. This makes Netrin-(1) an unexpected very specific target for imagery and/or targeted therapy. The present invention thus relates to compounds comprising an anti-Netrin-1 antibody, especially NP(137), a chelating moiety, optionally associated with a radioisotope, and their use either in imagery, diagnosis, especially companion diagnosis, or in targeted therapy. New diagnostic tests, which may be companion tests, and new cancer therapies, that may be combined to the companion test, are also proposed.

The present invention concerns a method and reagents to detect and locate or visualize Netrin-1 in tumors, as well as a method and agents to treat a cancer based on Netrin-1 presence. The present invention particularly concerns a new diagnostic test, which may be a companion test, and a new cancer therapy, that may be combined to the companion test.

### BACKGROUND OF INVENTION

There are a number of methods currently used to treat each type of cancer, including surgery, radiotherapy, chemotherapy, targeted therapy and immunotherapy. Successful cancer therapy is directed to the primary tumor and to any metastases, whether clinically apparent or microscopic.

It is of interest for the patient to identify as early as possible the presence of cancer and possibly localize the cancer and determine the type of cancer to be treated. Cancer that is diagnosed at an early stage, is more likely to be treated successfully. If cancer spreads, effective treatment becomes more difficult, and generally chances of surviving are much lower. So, it is essential to know when to use immediately a heavy and aggressive treatment protocol in order to prevent extension of an aggressive cancer.

Also, treatment protocols even if they are quite successful may left tumor cells or stem tumor cells in some places. It is also crucial to identify and localize these cells.

It may also be of interest for the patient to be able to propose anti-cancer targeted therapy. However, in case of targeted therapy, there is a real need for reagents allowing in vivo detection and localization of a cancer, and determination of some molecular signature of cancer, so that an appropriate targeted therapy can be provided at the earliest possible stage or as a complementary protocol only in those patients having a cancer eligible to said treatment.

Netrin-1 plays a major role in the development of organisms and more particularly in the establishment of the central nervous system. It thus possesses an attractive role for commissural neurons. Netrin-1 has been described for years in neural development as a secreted molecule with a diffusible grade. Signaling pathways are transduced by receptors called Deleted in Colorectal Carcinoma (DCC), uncoordinated-5 (UNC-5) family and Neogenin. All its molecular pathways imply that Netrin-1 is described as acting in a pleiotropic number of diseases or signaling mechanisms.

Netrin-1 has also been shown to be up-regulated in many cancer types such as breast, NSCLC, medulloblastoma. This over-expression by tumour cells has been proposed to act as a molecular mechanism that blocks cell death induced by the dependence receptors activities of DCC and Unc-5 family. These receptors act as tumor suppressor genes and trigger apoptosis in absence of their ligand. To counteract this safeguard mechanism, tumor cells activates netrin-1 expression, leading to an overexpression of this protein to inhibit cell death for example after chemotherapies. Reactivating this molecular mechanism thus appear as a therapeutic target in oncology. As a consequence, therapeutic strategies have been developed to block Netrin-1, and more precisely inhibit the interaction between Netrin-1 and its receptors on the surface of the cancer cells. A Phase I-II clinical trial began to evaluate a human IgG1 called NP137 (humanized monoclonal antibody) and capable of blocking the Unc5-B/netrin-1 interaction. Interim results show encouraging signs of clinical activity as a single agent. Netrin-1 blockage thus appears to be effective in a subset of patients but predicting patient benefit using approved simple companion test is lacking, all biopsy-based tests being subject to the errors and limitations of invasive tissue collection.

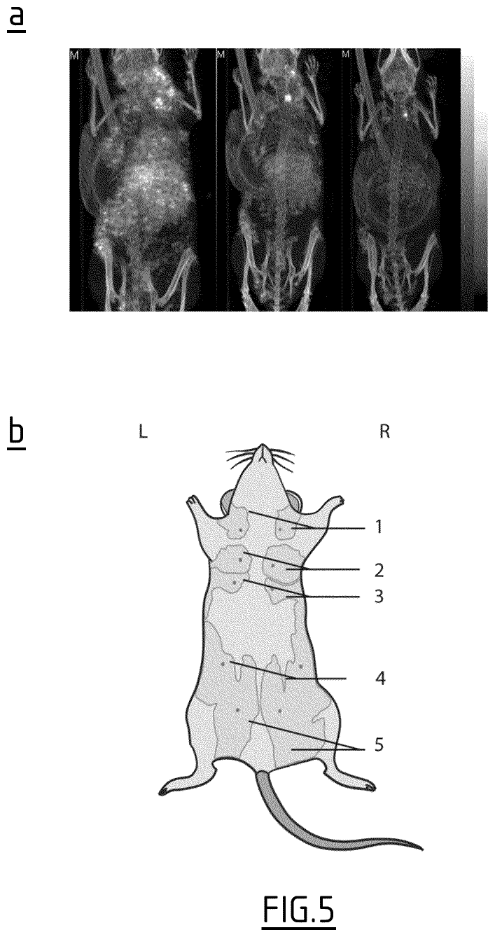

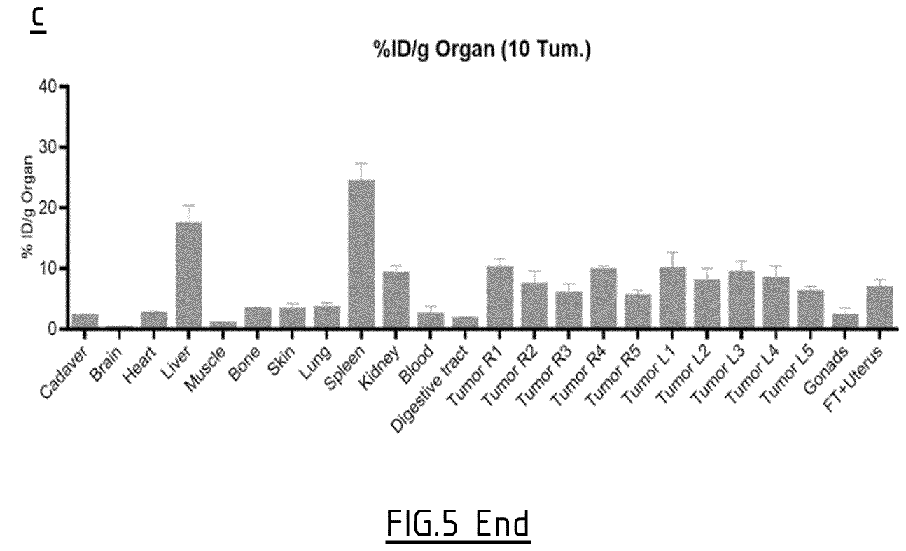

J. Wischhusen et al. (Theranostics 2018; 8(18): 5126-5142) disclose that netrin-1 co-localized with endothelial CD31 in netrin-1-positive breast tumors. Netrin-1 localized on the vascular endothelium of these tumors. Ultrasound molecular imaging (USMI) was proposed as a non-invasive companion diagnostic for netrin-1 interference therapy in breast cancer. Netrin-1 detected on the surface of endothelial cells is imaged with very short times (in the order of ten minutes) and allows the visualization of netrin-1 sequestered on the endothelial cells constituting the vessels. The results in terms of tumor incorporation are very low, with a background to incorporation ratio that is very low in FIG. 5A: from 32% to 45%, i.e., a real increase of 1.4×.

Radioactive imagery and internal radioactivity therapy are performed to target membranous receptors or surface molecules. Secreted factors or ligands that are generally considered more or less diffusible are generally not selected for these techniques.

J. Wischhusen et al. (infra) do not disclose netrin-1 sequestered in the cell matrix at the cell periphery of cancer cells, and the mere disclosure that netrin-1 is sequestered on the endothelial cells constituting the vessels does not qualify netrin-1 detection as a robust tool to detect and localize netrin-1 expressing tumors.

Netrin-1 which is mainly qualified as a secreted protein, or a protein sequestered on the surface of vascular endothelial cells, does not primarily appear to be a candidate for imagery and/or targeted therapy.

### SUMMARY OF INVENTION

Presented herein is the unexpected extensive demonstration that Netrin-1 is retained in a stickier manner in the cell matrix at the cell periphery of the cancer cells as revealed by the tumor accumulation of Netrin-1. Interestingly, Netrin-1, which is a protein expressed at the embryonic stage, is expressed in adults specifically in some tumors. Combined with the fact that Netrin-1 is retained at the tumor location in the extracellular cell matrix (ECM), this makes Netrin-1 an unexpected very specific target for imagery and/or targeted therapy. Sequestration of netrin-1 in the cell matrix of the tumor cells open the way to imaging methods with long acquisition times, such as from about 24 to about 96 h, which allows the visualization of netrin-1 sequestered in the extracellular matrix of the tumor itself, and thus a true and powerful imaging of the whole tumor, contrary to the USMI described in J. Wischhusen et al. For example, the background to incorporation ratio with a method such as SPECT may be high, e.g., of the order of 5.8×, as obtained on the 4T1 cells Unexpectedly also, it is shown herein that Netrin-1 is expressed very early during tumor formation and thus allows one to detect, localize and/or therapeutically target tumor cells expressing Netrin-1 very early, before appearance of a small lesion or before palpation, such as mammary palpation.

Aspects of the invention thus concern compounds per se, usable either in imagery, diagnosis, especially companion diagnosis, or in targeted therapy. At the basis is a compound comprising an anti-Netrin-1 antibody or antigen binding fragment thereof, and a chelating moiety bound to said antibody or fragment, wherein said chelating moiety is optionally associated with a radioisotope. The very isotope associated thereto may dictate the use of the compound, between imagery and targeted therapy.

### DETAILED DESCRIPTION

In a first aspect, the present invention relates to a compound comprising:
an anti-Netrin-1 antibody or antigen binding fragment thereof, and
a chelating moiety bound to said antibody or fragment,

wherein said chelating moiety is optionally associated with a radioisotope.

Typically, the antibody or fragment thereof and the chelating moiety are covalently linked. According to this embodiment, the present compound is a conjugate. In an embodiment, the chelating moiety binds to a side-chain of an amino acid of the antibody or fragment thereof, especially a side-chain residue of a Lysine.

Typically, the radioisotope is bound to the chelating moiety by a covalent bond.

The compounds of the invention are particularly useful because they are capable of specifically binding to Netrin-1 in vivo, thus enabling the imaging of said cancer or its targeting by the binding of the antibody to netrin-1 in the cell matrix at the cell periphery of the cancer cells. This is particularly advantageous for identifying the localization of a cancer and/or follow cancer growth or regression. Notably, the radiolabeled compounds are used for flow visualization through different technologies, such as Single Photon Emission Computed Tomography (SPECT) and Positron Emission Tomography (PET). The radiolabeled compounds may also be used for radiation therapy, or both visualization and therapy.

### Antibody

The compound preferably comprises a monoclonal antibody (mAb) or an antigen-binding fragment thereof, wherein the mAb or its fragment specifically binds to Netrin-1. The mAb may be a murine, a chimeric, a humanized or a full-human monoclonal antibody. The fragment may be any type of mAb fragment that keeps substantially the ability of the whole antibody to bind to Netrin-1, it can be for example a Fab or a F(ab′)₂.

Examples of useful murine, chimeric and humanized monoclonal antibodies are disclosed in U.S. Pat. No. 10,494,427, which is incorporated herein by reference. Specific embodiments disclosed in this prior document and that can be used herein are the following antibodies listed in Table 1. The first listed in Table 1 corresponds to the murine 4C11 mAb, the second listed HUM00 corresponds to the grafting of the murine 4C11 CDRs into a human IgG1. The ten mAb HUM01 to HUM10 correspond to humanized mAbs derived from HUM00 with specific modifications in the FR regions of the human IgG. HUM03 is also called NP137. Sequences of the human IgG1 CH come from Genbank AEL33691.1 modified R97K. Sequences of the human IgG1 CL (Kappa) come from Genbank CAC20459.1. The other allotypes may be used as well. Specific binding of all these mAbs, Fab fragments and F(ab′)₂ fragments to Netrin-1 is demonstrated in US2018/0072800.

TABLE 1

| 4C11 | SEQ ID NO: 6 | | SEQ ID NO: 7 | |
|---|---|---|---|---|
| (murine) | VH SEQ | Constant heavy | VL SEQ | Constant light |
| Humanized | ID NO: | chain (CH) | ID NO: | chain (CL) |
| HUM00 | 21 | Human IgG1 | 13 | Human IgG1 |
| HUM01 | 14 | Human IgG1 | 8 | Human IgG1 |
| HUM02 | 15 | Human IgG1 | 9 | Human IgG1 |
| HUM03 | 16 | Human IgG1 | 10 | Human IgG1 |
| HUM04 | 17 | Human IgG1 | 11 | Human IgG1 |
| HUM05 | 18 | Human IgG1 | 11 | Human IgG1 |
| HUM06 | 19 | Human IgG1 | 10 | Human IgG1 |
| HUM07 | 20 | Human IgG1 | 11 | Human IgG1 |
| HUM08 | 16 | Human IgG1 | 11 | Human IgG1 |
| HUM09 | 19 | Human IgG1 | 12 | Human IgG1 |
| HUM10 | 15 | Human IgG1 | 10 | Human IgG1 |

In an embodiment, the antibody is a monoclonal antibody or an antigen-binding fragment thereof, comprising
a variable domain VH comprising:
   a H-CDR1 having a sequence set forth as SEQ ID NO: 1;
   a H-CDR2 having a sequence set forth as SEQ ID NO: 2;
   a H-CDR3 having a sequence set forth as SEQ ID NO: 3;
a variable domain VL comprising:
   a L-CDR1 having a sequence set forth as SEQ ID NO: 4;
   a L-CDR2 having a sequence YAS;
   a L-CDR3 having a sequence set forth as SEQ ID NO: 5;
or
a variable domain VH comprising:
   a H-CDR1 having a sequence set forth as SEQ ID NO: 22;
   a H-CDR2 having a sequence set forth as SEQ ID NO: 23;
   a H-CDR3 having a sequence set forth as SEQ ID NO: 24;
a variable domain VL comprising:
   a L-CDR1 having a sequence set forth as SEQ ID NO: 25;
   a L-CDR2 having a sequence set forth as SEQ ID NO: 26;
   a L-CDR3 having a sequence set forth as SEQ ID NO: 5.

Preferably, the antibody is a monoclonal antibody or an antigen-binding fragment thereof, comprising a pair of VH and VL sequences selected from the following pairs: SEQ ID NO: 21 and 13, SEQ ID NO: 14 and 8, SEQ ID NO: 15 and 9, SEQ ID NO: 16 and 10, SEQ ID NO: 17 and 11, SEQ ID NO: 18 and 11, SEQ ID NO: 19 and 10, SEQ ID NO: 20 and 11, SEQ ID NO: 16 and 11, SEQ ID NO: 19 and 12, SEQ ID NO: 15 and 10. More preferably, the antibody is a monoclonal antibody or an antigen-binding fragment thereof, comprising a pair of VH and VL sequences SEQ ID NO: 16 and 10.

The anti-netrin-1 antibody or an antigen-binding fragment thereof may further comprise a Human IgG1 Constant heavy chain (CH) and/or a Human IgG1 Constant light chain (CL). In an embodiment, sequences of the human IgG1 CH come from Genbank AEL33691.1 modified R97K. Sequences of the human IgG1 CL (Kappa) come from Genbank CAC20459.1. In an embodiment, the mAb is NP137 and comprises SEQ ID NO: 16 and 10 as VH, respectively VL sequences, and those specific IgG1 CH and CL.

TABLE 2
Description of the sequences:

| SEQ | | |
|---|---|---|
| ID | | |
| NO: | Description | Sequence |
| 1 | aa seq. of CDR1-H | GYTFTSYN |
| | (IMGT) | |
| 2 | aa seq. of CDR2-H | IYPGNGDT |
| | (IMGT) | |
| 3 | aa seq. of CDR3-H | ARGGTGFAY |
| | (IMGT) | |
| 4 | aa seq. of CDR1-L | QSVSND |
| | (IMGT) | |
| — | aa seq. of CDR2-L | YAS |
| | (IMGT) | |
| 5 | aa seq. of CDR3-L | QQDYSSPWT |
| | (IMGT and Kabat) | |
| 6 | Full aa sequence of | QAYLQQSGAELVRPGASVKMSCKASGYTFTSYNMH |
| | 4C11 (VH + mouse | WVKQTPRQGLEWIGAIYPGNGDTSYNQKFKGKATLT |
| | lgG1 CH) | VDKSSSTAYMQLSSLTSEDSAVYFCARGGTGFAYWG |
| | | QGTLVTVSAAKTTPPSVYPLAPGSAAQTNSMVTLGCL |
| | | VKGYFPEPVTVTWNSGSLSSGVHTFPAVLESDLYTLS |
| | | SSVTVPSSPRPSETVTCNVAHPASSTKVDKKIVPRDC |
| | | GCKPCICTVPEVSSVFIFPPKPKDVLTITLTPKVTCVVV |
| | | DISKDDPEVQFSWFVDDVEVHTAQTQPREEQFNSTF |
| | | RSVSELPIMHQDWLNGKEFKCRVNSAAFPAPIEKTISK |
| | | TKGRPKAPQVYTIPPPKEQMAKDKVSLTCMITDFFPE |
| | | DITVEWQWNGQPAENYKNTQPIMNTNGSYFVYSKLN |
| | | VQKSNWEAGNTFTCSVLHEGLHNHHTEKSLSHSPGK |
| 7 | Full aa sequence of | SIVMTQTPKFLLVSAGDRVTITCKASQSVSNDVAWYQ |
| | 4C11 (VL + mouse | QKPGQSPKLLIYYASNRYTGVPDRFTGSGYGTDFTFT |
| | Kappa CL) | ISTVQAEDLAVYFCQQDYSSPWTFGGGTKLEIKRADA |
| | | APTVSIFPPSSEQLTSGGASVVCFLNNFYPKDINVKW |
| | | KIDGSERQNGVLNSWTDQDSKDSTYSMSSTLTLTKD |
| | | EYERHNSYTCEATHKTSTSPIVKSFNRNEC |
| 8 | VL aa sequence of | EIVMTQSPATLSVSPGERATLSCKASQSVSNDVAWY |
| | humanized variant of | QQKPGKAPKLLIYYASNRYTGIPPRFSGSGYGTDFTL |
| | 4C11 | TINNIESEDAAYYFCQQDYSSPWTFGQG |
| 9 | VL aa sequence of | DIQMTQSPSSLSASVGDRVTITCKASQSVSNDVAWFQ |
| | humanized variant of | QRPGQSPRRLIYYASNRYTGVPSRFSGSGSGTDFTF |
| | 4C11 | TISSLEAEDAATYYCQQDYSSPWTFGQG |
| 10 | VL aa sequence of | DIQMTQSPSSLSASVGDRVTITCKASQSVSNDVAWY |
| | humanized variant of | QQKPGQAPRLLIYYASNRYTGIPPRFSGSGYGTDFTL |
| | 4C11 | TINNIESEDAAYYFCQQDYSSPWTFGQG |
| 11 | VL aa sequence of | DIQMTQSPSSLSASVGDRVTITCKASQSVSNDVAWYL |
| | humanized variant of | QKPGQSPQLLIYYASNRYTGVPSRFSGSGSGTDFTFT |
| | 4C11 | ISSLEAEDAATYYCQQDYSSPWTFGQG |
| 12 | VL aa sequence of | DIVMTQTPLSLPVTPGEPASISCKASQSVSNDVAWYQ |
| | humanized variant of | QKPGQAPRLLIYYASNRYTGIPPRFSGSGYGTDFTLTI |
| | 4C11 | NNIESEDAAYYFCQQDYSSPWTFGQG |
| 13 | VL aa sequence of | EIVMTQSPATLSVSPGERATLSCRASQSVSNDVAWY |
| | humanized variant of | QQKPGQAPRLLIYYASNRYTGIPARFSGSGSGTEFTL |
| | 4C11 | TISSLQSEDFAVYYCQQDYSSPWTFGQG |
| 14 | VH aa sequence of | QVQLVQSGAEVKKPGASVKVSCKASGYTFTSYNMH |
| | humanized variant of | WVRQATGQGLEWMGAIYPGNGDTSYNQKFKGRVTIT |
| | 4C11 | ADKSTSTAYMELSSLRSEDTAVYYCARGGTGFAYWG |
| | | QG |
| 15 | VH aa sequence of | QVQLQQSGPGLVKPSQTLSLTCAISGYTFTSYNMHWI |
| | humanized variant of | RQPPGKGLEWIGAIYPGNGDTSYNQKFKGRVTITADK |
| | 4C11 | STSTAYMELSSLRSEDTAVYYCARGGTGFAYWGQG |
| 16 | VH aa sequence of | QVQLQQSGPGLVKPSQTLSLTCAISGYTFTSYNMHW |
| | humanized variant of | VRQATGQGLEWMGAIYPGNGDTSYNQKFKGRLTISK |
| | 4C11 | DTSKNQVVLTMTNMDPVDTATYYCARGGTGFAYWG |
| | | QG |
| 17 | VH aa sequence of | EVQLVQSGAEVKKPGESLRISCKGSGYTFTSYNMHW |
| | humanized variant of | VRQATGQGLEWMGAIYPGNGDTSYNQKFKGRFTISR |
| | 4C11 | DDSKNTAYLQMNSLKTEDTAVYYCARGGTGFAYWG |
| | | QG |
| 18 | VH aa sequence of | QVQLQESGPGLVKPSQTLSLTCTVSGYTFTSYNMHW |
| | humanized variant of | VRQAPGQGLEWMGAIYPGNGDTSYNQKFKGRVTISV |
| | 4C11 | DTSKNQFSLKLSSVTAADTAVYYCARGGTGFAYWGQ |
| | | G |
| 19 | VH aa sequence of | QVQLVQSGAEVKKPGASVKVSCKASGYTFTSYNMH |
| | humanized variant of | WVRQATGQGLEWMGAIYPGNGDTSYNQKFKGRVTIT |
| | 4C11 | ADKSTSTAYMELSSLRSEDTAVYYCARGGTGFAYWG |
| | | QG |
| 20 | VH aa sequence of | QVQLQQSGPGLVKPSQTLSLTCAISGYTFTSYNMHW |
| | humanized variant of | VRQATGQGLEWMGAIYPGNGDTSYNQKFKGRVTITA |
| | 4C11 | DKSTSTAYMELSSLRSEDTAVYYCARGGTGFAYWGQ |
| | | G |
| 21 | VH aa sequence of | QVQLVQSGAEVKKPGASVKVSCKASGYTFTSYNMH |
| | humanized variant of | WVRQAPGQGLEWMGAIYPGNGDTSYAQKFKGRVTM |
| | 4C11 | TRDTSTSTVYMELSSLRSEDTAVYYCARGGTGFAYW |
| | | GQ |
| 22 | aa seq. of CDR1-H | SYNMH |
| | (Kabat) | |
| 23 | aa seq. of CDR2-H | AIYPGNGDTSYNQKFKG |
| | (Kabat) | |
| 24 | aa seq. of CDR3-H | GGTGFAY |
| | (Kabat) | |
| 25 | aa seq. of CDR1-L | KASQSVSNDVA |
| | (Kabat) | |
| 26 | aa seq. of CDR2-L | YASNRYT |
| | (Kabat) | |

CDRs under IMGT are highlighted in bold in Table 1 where appropriate.

As anti-netrin-1 antibodies that may be used, one may cite other antibodies, especially monoclonal antibodies, or their antigen-binding fragments, developed against human netrin-1 or against animal netrin-1, netrin-1 being very homologous among species. May be cited: Abcam antibodies ab126729, ab122903, ab201324, ab39370; AF1109, AF6419, AFR 28.

### Chelating Moiety:

A “chelating moiety” or “chelating agent” or “chelator” as used herein refers to a compound which is capable of chelating any of the radioisotopes. The chelating moiety sequesters the corresponding free radioisotopes generally from aqueous solutions, thus enabling applying said isotopes to specific biological applications. Said chelating moiety is a bifunctional chelator. A “bifunctional chelator or “bifunctional chelating agent” as used herein refers to a compound possessing a metal binding moiety function and a chemically reactive functional group allowing binding to the antibody.

Numerous bifunctional chelators are known in the art. A great number of them are indeed available commercially and have been routinely used as PET imaging agents. Example of bifunctional chelating agents are: NODAGA (1,4,7-triazacyclononane-1-glutaric acid-4,7-diacetic acid), DOTA (1,4,7,10-tetraazacyclododecane-1,4,7,10-tetraacetic acid), p-SCN-Bn-NOTA, p-SCN-Bn-PCTA, p-SCN-Bn-oxo-DO3A, desferrioxamine-p-SCN, Diethylenetriamine Pentaacetic Acid (DTPA), 1,4,8,11-Tetraazacyclotetradecane-1,4,8,11-tetraacetic acid (TETA). NOTA (4,7-triazacyclononane-1,4,7-triacetic acid.

The bifunctional chelator is preferably an ester of these chelating agents. Preferably, the chelator is NODAGA-NHS (NODAGA N-hydroxysuccinimide ester) or DOTA-NHS (DOTA N-hydroxysuccinimide ester).

### Radioisotope:

A “radioisotope” as used herein is a version of a chemical element that has an unstable nucleus and emits radiation during its decay to a more stable or a stable form. The radioisotope of the present compounds may be those employed in imagery or in radionuclide therapy.

Radioisotopes useful in the invention comprise in particular ⁶⁸Ga, ⁶⁴Cu, ⁸⁹Zr, ¹⁸⁶Re, ¹⁸⁸Re, ¹⁵³Sm, ¹¹¹In, ^{99m}Tc, ¹²³I, ¹⁷⁷Lu, ⁹⁰Y, ¹³¹I, ²¹³Bi, ²¹²Bi, ²¹¹At, ²²⁵Ac.

For imagery, one may mention more particularly ⁶⁸Ga, ⁶⁴Cu, ⁸⁹Zr, ¹⁸⁶Re, ¹⁸⁸Re, ¹⁵³Sm, ¹¹¹In, ^{99m}Tc, ¹²³I.

For therapy, the radionuclide may be selected more particularly from those used in internal radioactivity therapy, which are metals inducer of cytotoxicity. May be used the beta-emitting radionuclides such as lutetium-177 (¹⁷⁷Lu), yttrium-90 (⁹⁰Y), and iodine-131 (¹³¹I). May also be used alpha-emitting radionuclides such as Bismuth-213 (²¹³Bi), Bismuth-212 (²¹²Bi), Astatine-211 (²¹¹At) and Actinium-225 (²²⁵Ac).

These radioisotopes are preferably chosen in consideration of their half-life, which is preferably long, which makes them particularly suitable for in vivo use, e.g., PET/SPECT imaging or targeted radiotherapy.

### Compound and Composition:

One or several, e.g., 2 to 10, chelator or chelating moieties may bind to the one antibody. Thus, the compound of the invention may comprise:
an anti-Netrin-1 antibody or antigen binding fragment thereof, and
one or several, especially 2 to 10, chelating moieties, bound to said antibody or fragment,

wherein said chelating moieties are optionally associated with a radioisotope. In an embodiment, one or more of the chelating moieties are associated with a radioisotope. The anti-Netrin-1 antibody or antigen binding fragment thereof may be any of the above-described monoclonal antibodies or antigen-binding fragments thereof. In a particular embodiment, the antibody is NP137.

Another aspect of the invention is a composition comprising such a compound which comprises:
an anti-Netrin-1 antibody or antigen binding fragment thereof, and
one or several, especially 2 to 10, chelating moieties, bound to said antibody or fragment,

wherein said chelating moieties are associated with a radioisotope,

and a pharmaceutically acceptable vehicle. In an embodiment, one or more chelating moieties bound to an antibody are associated with a radioisotope.

In an embodiment, the composition may comprise an anti-Netrin-1 antibody or antigen binding fragment thereof to which no chelating moiety is bound.

These compounds and compositions may be prepared using well-known methods, such as those disclosed herein.

These compositions may further comprise a pharmaceutically acceptable carrier or vehicle.

### Preparation of the Compound:

In another aspect, the present invention provides a method of preparing the compound of the invention. Said method comprises the steps of:
a) conjugating a chelating moiety to the antibody or fragment thereof; and
b) recovering the conjugate of antibody or fragment thereof and chelator.

Conjugating is obtained by incubating the amine-reactive chelating moiety with the antibody or fragment thereof. Incubation is made for a duration that is enough to obtain chelation. Typically, the duration is of about 5 minutes to about 2 hours. Incubation is made at a temperature that is not denaturing for the antibody or its fragment. Temperature may typically be comprised between about 35 and about 42° C., preferably from about 37 to about 40° C.

Amine-reactive chelate structures for the radioisotope described herein are commercially available, such as e.g., DOTA-NHS and NODAGA-NHS esters. It is deemed the NHS esters (N-hydroxysuccinimide esters) will react with primary amines at the N-terminus and in the side-chain of lysine (Lys, K) amino acid residues of the antibody, as this occurs with peptides. The binding thus needs not to be detailed here.

One or several, e.g., 2 to 10, chelator moieties may bind to the one antibody comprising a number of lysine amino acids.

Preferably, the method of preparing the compound of the invention further comprises a step of:
c) incubating the conjugate of antibody or fragment thereof and chelator with the complementary radioisotope;

thus, generating the compound of the invention. The compound may then be recovered and formulated in a pharmaceutical carrier or vehicle.

Incubation c) is made for a duration that is enough to ensure the radioisotope binding. Typically, the duration is of about 5 minutes to about 2 hours. Incubation is made at a temperature that is not denaturing for the antibody or its fragment. Temperature may typically be comprised between about 35 and about 42° C., preferably from about 37 to about 40° C.

### Imaging

In another aspect, the present invention provides a method of imaging Netrin-1 presence or localization in a subject or of imaging Netrin-1 presence or accumulation in organs or tissues, or of imaging Netrin-1 expressing cancer, by administering to an organism (an animal, in particular a mammal, especially a human) an effective amount of the compound, where the compound includes a metal isotope suitable for imaging.

In another aspect, the present invention relates to a compound comprising an anti-Netrin-1 antibody or antigen-binding fragment thereof, a chelating moiety bound to said antibody or fragment, and a radioisotope associated to the chelating moiety, for use in the in vivo imaging of a cancer. Advantageously, netrin-1 is detected in the cell matrix at the periphery of the cancer cells, wherein netrin-1 is accumulated.

In an embodiment, imaging gives an information on a relative level of netrin-1 presence or expression in the detected area (e.g., organ or tissue) owing the compound of the invention.

“Accumulation of Netrin-1” means in particular the accumulation of netrin-1 in the cell matrix at the cancerous cell periphery. Thus, netrin-1 may be present and accumulated in tissues or organs, in the vicinity or surrounding environment of cancer cells or tumors.

Imaging can be performed by any suitable technique known to the person skilled in the art, allowing detecting and/or visualizing, notably PET or SPECT, especially coupled to CT scanners (computerized tomography). A radionuclide, such as a one produced from either a cyclotron or a generator, is attached to a biologically active molecule forming a radiotracer, e.g., a SPECT or PET radiotracer. In the present case, the molecule is the compound made of the antibody or fragment thereof and the chelator, and the binding thereto of a radionuclide constitutes the radiotracer. The radiotracer is then introduced into the patient, preferably by injection, such as intravenous (IV) injection.

According to an aspect of the invention, there is provided a method for imaging Netrin-1 presence or localization (e.g., visualization) in a subject, comprising:
a) administering, preferably injecting, a compound as described herein, to said subject;
b) detecting or localizing said compound by in vivo imaging, preferably PET or SPECT imaging.

According to an aspect of the invention, there is provided a method for cancer detection and localization in a subject, comprising:
a) administering to said subject a compound comprising:
   an anti-Netrin-1 antibody or antigen binding fragment thereof,
   a chelating moiety bound to said antibody or fragment, and
   a radioisotope associated to the chelating moiety,
b) detecting and localizing said cancer by in vivo imaging in the cell matrix at the periphery of the cancer cells.

In an aspect, before detecting or localizing, a time lapse is respected between steps a) and b), this is a time or acquisition time, such as from about 4 to about 172 h, in particular about 12 to about 172 h, preferably from about 24 to about 96 h, or about 24 to about 48 h, which allows the binding of said compound by the netrin-1 sequestered in the extracellular matrix. More precisely, this is a time enough to allow the administered compound to leave the blood circulation, penetrate the tumor or the tumors, and reach the netrin-1 sequestered in the tumor cell matrix. This allows the following step of detecting or localizing said bound compound by in vivo imaging.

At step b) or in the “use for”, the in vivo imaging detects or highlights the presence or accumulation of the compound in at least one body part, e.g., organ or tissue. This presence or accumulation is specific in the sense that the compound binds to netrin-1 accumulated into said body part. It is specific as there is a time laps between compound administration and imaging.

Latency or time laps between administration and detection is chosen so that detection or imaging is performed at a time the antibody or fragment thereof is specifically bound to netrin-1. Indeed, after administration, there is a phase of spread of the compound in the body and its organs, and only after some time the presence of the compound in an organ or part of the body is specific of the presence of netrin-1 and of the binding of the compound thereto. The time laps may be of the order of from about 4 hours to about 168 hours; typically, about 4 hours to about 96 hours. In practice, the time laps retained shall be compatible with the half-life of the radioisotope, and conversely.

According to another aspect of the invention, there is thus provided a compound as described herein, for use in as an imaging radioisotope compound. This use is in particular intended to imaging Netrin-1 presence or localization in a subject, as explained above. The compound is in particular for use in an in vivo imaging, preferably PET or SPECT imaging.

In an embodiment, the method or use provides an image of a part of the body, in particular an image of an organ or tissue or subpart thereof (e.g., lung, pancreas, bladder, spleen, kidney, stomach, colon, small intestine, intestine, esophagus, muscle, skin, brain) and optionally the surrounding tissues or organs.

In an embodiment, the method or use provides an image of an anatomical part of the body, in particular an image of a leg, an arm, the chest, abdomen, head, and subparts thereof.

In an embodiment, the method or use provides for an image of the whole body.

In PET, the system detects pairs of gamma rays emitted indirectly by a radionuclide (tracer), which is introduced into the body on the radiotracer. Three-dimensional images of tracer concentration within the body are then constructed by computer analysis. In modern PET-CT scanners, three-dimensional imaging is often accomplished with the aid of a CT X-ray scan performed on the patient during the same session, in the same machine.

In PET, the standard uptake value may be calculated allowing to obtain quantification of the tracer in an observed area (e.g., tissue or organ). This may allow generating a certain quantification of netrin-1 presence or expression in the observed area. As an alternative, the radiologist has the skill to notice an accumulation of the tracer in an area by simple observation, which accumulation is distinguishable from the background noise. This is called “positive accumulation detection” herein.

Single-photon emission computed tomography (SPECT) is a nuclear medicine imaging technique similar to PET. It also uses a radioactively labelled tracer and is based on the detection of gamma rays. In contrast to PET, the radioactive label used in SPECT emits a gamma radiation that is measured directly. Combined with a CT scanner, the SPECT-CT provides for three-dimensional imaging as well.

SPECT imaging may be exploited with comparison between the observed area (e.g. tissue or organ) and the liver. This may allow generating a result defined as higher, equal or below the liver level. The radiologist has the skill to notice an accumulation of the tracer in an area by simple observation, which accumulation is distinguishable from the background noise. This is called “positive accumulation detection” herein.

Due to the short half-lives of most positron-emitting radioisotopes, the radiotracers have traditionally been produced using a cyclotron in close proximity to the PET or SPECT imaging facility. The half-life of fluorine-18 is long enough that radiotracers labelled with fluorine-18 can be manufactured commercially at offsite locations and shipped to imaging centers. On the other hand, ⁶⁸Ga can be produced in a generator, thus disposing with the need of a cyclotron. In addition, the half-life of gallium-68 is close to the one of ¹⁸F, making this radionuclide particularly useful for PET imaging.

In an embodiment ¹¹¹In is used as the radionuclide. During its radioactive decay, it emits low energy gamma (γ) photons and its half-life is 2.8 days. It is generally produced in a cyclotron. Its half-life is long enough that radiotracers labelled with it can be manufactured commercially at offsite locations and shipped to imaging centers.

The imaging method is suitable for detecting and localizing Netrin-1 in a cancerous tissue, a cancerous organ or in a body of a cancerous subject. As used herein, the term “cancer” refers to or describes the physiological condition in mammals that is typically characterized by unregulated cell proliferation. The terms “cancer” and “cancerous” as used herein are meant to encompass all stages of the disease. A “cancer” as used herein is any malignant neoplasm resulting from the undesired growth, the invasion, and under certain conditions metastasis of impaired cells in an organism. The cells giving rise to cancer are genetically impaired and have usually lost their ability to control cell division, cell migration behavior, differentiation status and/or cell death machinery. A cancer generally forms at a primary site, giving rise to a primary cancer. Cancer that spreads locally, or to distant parts of the body is called a metastasis. The imaging method herein will detect and localize those solid cancers, at any stage, expressing Netrin-1.

The compounds of the invention are also useful for diagnosing a cancer in a patient. According to this aspect, the invention provides a method of diagnosis of a cancer in a patient, said method comprising the steps of:
a) administering a compound as described herein, or a pharmaceutically acceptable salt thereof, to said subject;
b) detecting or localizing said compound by in vivo imaging, preferably PET or SPECT imaging; and
c) diagnosing a cancer based on step b).

At step b), the in vivo imaging detects or highlights the presence or accumulation of the compound in at least one body part, e.g., organ or tissue. This presence or accumulation is specific in the sense that the compound binds to netrin-1 accumulated into said body part. It is specific as there is a time laps between compound administration and imaging, as disclosed above.

According to another aspect of the invention, there is thus provided a compound as described herein, for use as an imaging diagnostic radio-isotope compound. This use may be intended to in vivo imaging Netrin-1 presence or localization in a subject, as explained above. It may help making diagnosis of a cancer. The compound is in particular for use in an in vivo imaging, preferably PET or SPECT imaging.

The present antibodies or fragments thereof only bind Netrin-1. Any signal detected in PET or SPECT imaging is thus an indication that Netrin-1 is present. Because of the accumulation of Netrin-1 in the cell matrix at the cancerous cell periphery and the sensitivity of the present radiolabeled compounds, it is possible to identify cancerous cells within the body of the patient, and thus diagnose a cancer, confirm a cancer, localize a cancer and/or identify the type of cancer. The type of cancer includes the name of the organ or tissue that is cancerous.

In another aspect, the present invention relates to a method of prognosis of a cancer in a patient, said method comprising the steps of:
a) administering a compound as described herein, or a pharmaceutically acceptable salt thereof, to said subject;
b) detecting said compound by in vivo imaging, preferably PET or SPECT imaging; and
c) prognosing a cancer based on the detection of step c).

At step b), the in vivo imaging detects or highlights the presence or accumulation of the compound in at least one body part, e.g., organ or tissue. This presence or accumulation is specific in the sense that the compound binds to netrin-1 accumulated into said body part. It is specific as there is a time laps between compound administration and imaging, as disclosed above.

This method comprises the further step of making a medical prognosis, for example in a patient that is or has been treated in accordance with an anticancerous therapy.

According to another aspect of the invention, there is thus provided a compound as described herein, for use in as an imaging prognosis radio-isotope compound. This use is in particular intended to in vivo imaging Netrin-1 presence or localization in a subject, as explained above, and prognosing cancer. The compound is in particular for use in an in vivo imaging, preferably PET or SPECT imaging.

“Prognosis” as used herein means the likelihood of recovery from a disease or the prediction of the probable development or outcome of a disease. For example, the bigger the single detected in step b), the bigger the cancerous mass in the patient's body, the worse the prognosis.

In yet another aspect, the present invention provides a method of determining the localization of a cancer in a subject in need thereof, comprising:
a) administering a compound as described herein, or a pharmaceutically acceptable salt thereof, to said subject;
b) detecting said compound by in vivo imaging, preferably PET or SPECT imaging;
c) visualizing localization of Netrin-1 presence or accumulation.

At step b), the in vivo imaging highlights the presence or accumulation of the compound in at least one body part, e.g., organ or tissue, that is visualized at step c). At step c) the body part, e.g., organ or tissue, is visualized and determined as comprising a netrin-1 presence or accumulation. If there is a visualized presence or accumulation of netrin-1 in this body part, there is strong presumption of cancer in it. This can be the discovery of cancer in said patient, or the identification of the body cancerous part, or both at the same time. This presence or accumulation is specific in the sense that the compound binds to netrin-1 accumulated into said body part. It is specific as there is a time laps between compound administration and imaging, as disclosed above.

According to another aspect of the invention, there is thus provided a compound as described herein, for use in as an imaging radio-isotope compound. This use is in particular intended to in vivo imaging Netrin-1 presence or localization in a subject, as explained above. The compound is in particular for use in an in vivo imaging, preferably PET or SPECT imaging. It is intended detecting netrin-1 in the cell matrix at the cell periphery of the cancer cells.

This method or use may further comprise the step of assessing the presence of a cancer in a given tissue or organ, or in several tissues and/or organs, the presence being evidenced by the Netrin-1 presence or accumulation.

It will be immediately clear to the skilled person that the invention also enables to identify the localization of a cancer at the earliest stages. Notably, the present invention is particularly useful for identifying the site of a cancer which is too small to be detected otherwise.

The pharmaceutical composition for imaging or a unit dosage form thereof comprises an effective amount of a compound described above. The present composition or unit dosage form may contain from about 5 to about 3 GBq, in particular 10 to 500 MBq, of the radionuclide-labelled imaging compound described above, in combination with a pharmaceutically acceptable carrier. The methods of use mentioned above may comprise the administration to a patient, especially a human one, a composition or unit dosage form comprising from about 0.1 mCi to about 100 mCi of the radionuclide-labelled imaging compound described above.

### Treating

According to another aspect, there is provided a method for treating a Netrin-1 expressing cancer in a subject in need thereof, comprising administering a therapeutically effective amount of a compound as described herein, to said subject. This method may be qualified of internal radioactivity therapy.

According to another aspect, there is provided such a compound as described herein for use in the treatment of a Netrin-1 expressing cancer in a subject.

The compound comprises an antibody or fragment thereof, which antibody or fragment specifically binds to Netrin-1, conjugated to a chelator moiety to which a radionuclide is associated. The radionuclide may be selected from those usual in internal radioactivity therapy, which are metals inducer of cytotoxicity. May be used the usual beta-emitting radionuclides such as lutetium-177 (¹⁷Lu), yttrium-90 (⁹⁰Y), and iodine-131 (¹³¹I). May also be used alpha-emitting radionuclides such as Bismuth-213 (²¹³Bi), Bismuth-212 (²¹²Bi), Astatine-211 (²¹¹At) and Actinium-225 (²²⁵Ac).

According to another aspect, there is provided such a compound as described herein for treating a Netrin-1 expressing cancer in a subject.

In an embodiment, ¹⁷Lu is used as the radionuclide. It is a gamma and beta emitter and its half-life is 6.7 days. It is generally produced in a cyclotron. Its half-life is long enough that radiotherapeutics labelled with it can be manufactured commercially at offsite locations and shipped to treating centers.

In an embodiment, ²²⁵Ac is used as the radionuclide. It is an alpha particle-emitting radionuclide that generates 4 net alpha particle isotopes in a short decay chain to stable ²⁰⁹Bi, and as such can be described as an alpha particle nanogenerator. It has a ten-day half-life. For more information, the skilled person may refer to M. Miederer et al. in Adv Drug Deliv Rev. 2008; 60(12): 1371-1382.

The compound is delivered to the patient by conventional route, preferably by parental route, e.g., by injection.

In an embodiment, the method or use is used to treat a patient identified positive for a cancer expressing Netrin-1. In particular, the patient was identified using the imaging method disclosed herein.

The pharmaceutical composition for therapy or a unit dosage form thereof comprises an effective amount of a compound described above. The present composition or unit dosage form may contain from about 5 to about 1000 MBq, in particular 10 to 500 MBq, of the radionuclide-labelled imaging compound described above, in combination with a pharmaceutically acceptable carrier.

### Imaging (Diagnosing) and Treating:

Where appropriate, the characteristics previously presented for “Imaging” and for “Treating” apply to “Imaging and Treating”.

According to another aspect, there is provided a method of identifying patients with cancer eligible for treatment with a monoclonal antibody or fragment thereof, said antibody or fragment thereof being able to inhibit interaction of Netrin-1 and its receptors on the surface of the cancer cells, comprising:
a) administering a compound as described herein, to said subject;
b) detecting said compound by in vivo imaging, preferably PET or SPECT imaging;
c) visualizing localization of Netrin-1 presence or accumulation;
d) treating said patient against the visualized cancer.

According to another aspect, there is provided a method of identifying patients with cancer eligible for treatment with a targeted radiotherapy, preferably comprising:
a) administering a compound as described herein, to said subject;
b) detecting said compound by in vivo imaging, preferably PET or SPECT imaging;
c) visualizing localization of Netrin-1 presence or accumulation;
d) treating said patient against the visualized cancer.

According to another aspect, there is provided a method of treating a cancer expressing Netrin-1, comprising:
a) administering a compound for imagery as described herein, to said subject;
b) detecting said compound by in vivo imaging, preferably PET or SPECT imaging;
c) visualizing localization of Netrin-1 presence or accumulation;
d) treating said patient against the visualized cancer.

In these methods, there is a further step between steps a) and b), which comprises waiting for the acquisition time mentioned above, in particular from 4 to 172 h, preferably from 24 to 96 h, obtaining binding of said compound by the netrin-1 sequestered in the extracellular matrix of the tumor.

In these different aspects, the compound administered at step a) is one of the compounds as described herein for in vivo imaging.

In these different aspects, treating at step d) may be made with existing anticancer treatments. However, in a preferred embodiment, the treating is made with a treatment that will specifically target the netrin-1 expressing cancer. This treatment may thus be made by administering an effective amount of an anti-netrin-1 antibody as disclosed in U.S. Pat. No. 10,494,427. The antibody may be one of the monoclonal antibodies disclosed herein in table 1, especially the so-called NP137. The method comprises administering a therapeutically effective amount of said mAb or fragment thereof. For the administration of those antibodies, the skilled person may refer to the mentioned US patent.

In an embodiment, therapy is internal radioactivity therapy as disclosed above. Thus, the therapy comprises administering a therapeutically effective amount of a compound as described herein, to said subject. The compound comprises an antibody or fragment thereof, which antibody or fragment specifically binds to Netrin-1, conjugated to a chelator moiety to which a radionuclide is associated. Said antibody may be one of the monoclonal antibodies disclosed herein in table 1, especially the so-called NP137. The antibody or fragment being conjugated to a chelating moiety which is itself bound to a radionuclide, as explained and detailed herein. The compound is intended to bind to netrin-1 in the tumor, including the netrin-1 sequestered in the cell matrix, and the radiotherapy may exerts its effect on the surrounding tumor cells or the whole tumor.

In an embodiment ¹¹¹In is used as the radionuclide associated with the compound for imaging.

In an embodiment, ¹⁷⁷Lu or ²²⁵Ac is used as the radionuclide associated with the compound for internal radioactivity therapy.

Doses of the imaging compound and of the therapy compound are as disclosed above.

### Formulation

The compositions of the invention can be formulated as a pharmaceutical composition, which comprises a compound of the invention and a pharmaceutically acceptable carrier. By a “pharmaceutically acceptable carrier” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimise any degradation of the active ingredient and to minimise any adverse side effects in the subject, as would be well known to one of skill in the art. For a discussion of pharmaceutically acceptable carriers and other components of pharmaceutical compositions, see, e.g., Remington's Pharmaceutical Sciences, 18th ed., Mack Publishing Company, 1990. Some suitable pharmaceutical carriers will be evident to a skilled worker and include, e.g., water (including sterile and/or deionized water), suitable buffers (such as PBS), physiological saline, cell culture medium (such as DMEM), artificial cerebral spinal fluid, or the like.

Dosages for compositions of the disclosure can be in unit dosage form. The term “unit dosage form” as used herein refers to physically discrete units suitable as unitary dosages for animal (e.g., human) subjects, each unit containing a predetermined quantity of a compound of the invention, calculated in an amount sufficient to produce the desired effect in association with a pharmaceutically acceptable diluent, carrier, or vehicle. One skilled in the art can easily determine the appropriate dose, schedule, and method of administration for the exact formulation of the composition being used, in order to achieve the desired effective amount or effective concentration of the agent in the individual patient.

For imagery, the dose of a composition described herein, administered to an animal, particularly a human, should be sufficient to produce at least a detectable amount of a diagnostic response in the individual over a reasonable time frame. The size of the dose will be determined by the existence of any adverse side effects that may accompany the particular agent, or composition thereof, employed. It is generally desirable, whenever possible, to keep adverse side effects to a minimum.

For therapy, the dose of a composition described herein, administered to an animal, particularly a human, should be sufficient to produce at least a detectable amount of cancer cell cytotoxicity, cancer cell death, cancer growth reduction or regression in the individual over a reasonable time frame. The size of the dose will be determined by the existence of any adverse side effects that may accompany the particular agent, or composition thereof, employed. It is generally desirable, whenever possible, to keep adverse side effects to a minimum.

The pharmaceutical or radiopharmaceutical composition may be administered parenterally, i.e., by injection, and is most preferably an aqueous solution. A “pharmaceutically acceptable carrier” refers to a biocompatible solution, having due regard to sterility, pH, isotonicity, stability, and the like and can include any and all solvents, diluents (including sterile saline, Sodium Chloride Injection, Ringer's Injection, Dextrose Injection, Dextrose and Sodium Chloride Injection, Lactated Ringer's Injection and other aqueous buffer solutions), dispersion media, coatings, antibacterial and antifungal agents, isotonic agents, and the like. The pharmaceutically acceptable carrier may also contain stabilizers, preservatives, antioxidants, or other additives, which are well known to one of skill in the art, or other vehicle as known in the art.

The present invention will now be described in more detail using non-limiting examples referring to the drawing comprising:

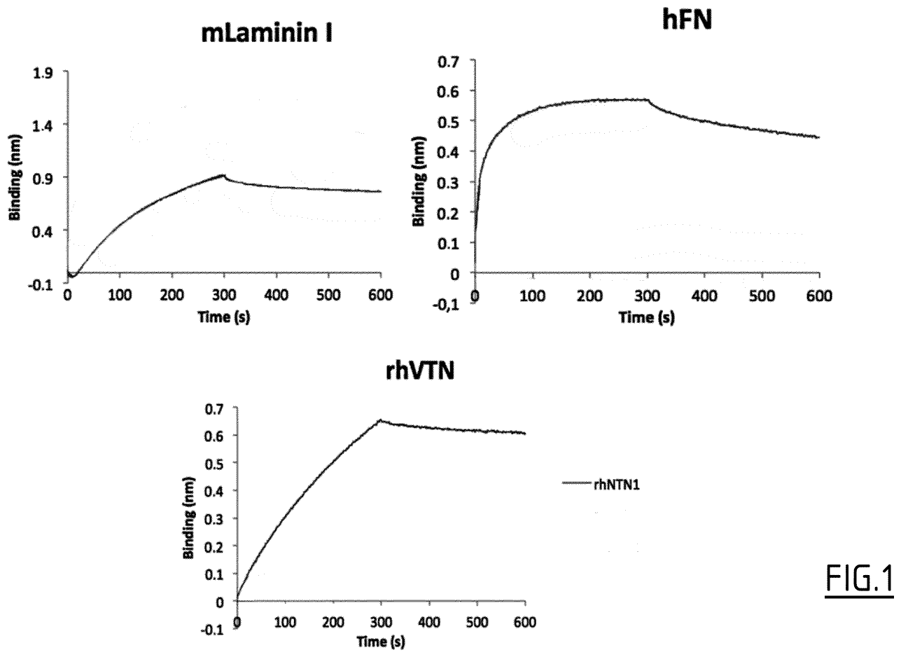

FIG. 1: Netrin-1 binds and is retain within the extracellular matrix. Analysis of h-netrin-1 (human recombinant netrin-1) binding on extra cellular matrix components: recombinant mouse laminin (m-Laminin), recombinant human fibronectin (h-fibronectin) and recombinant human Vitronectin (h-Vitronectin) by bio-layer interferometry assays.

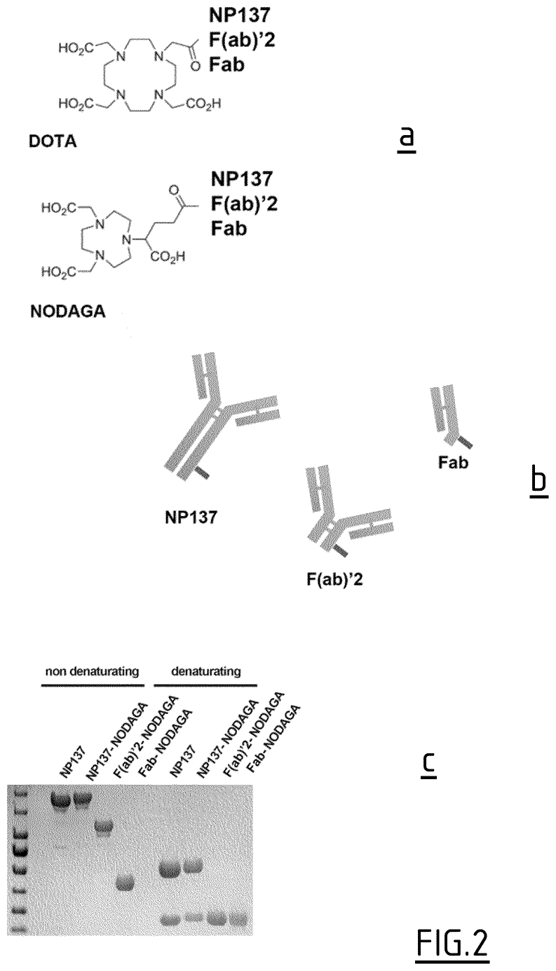

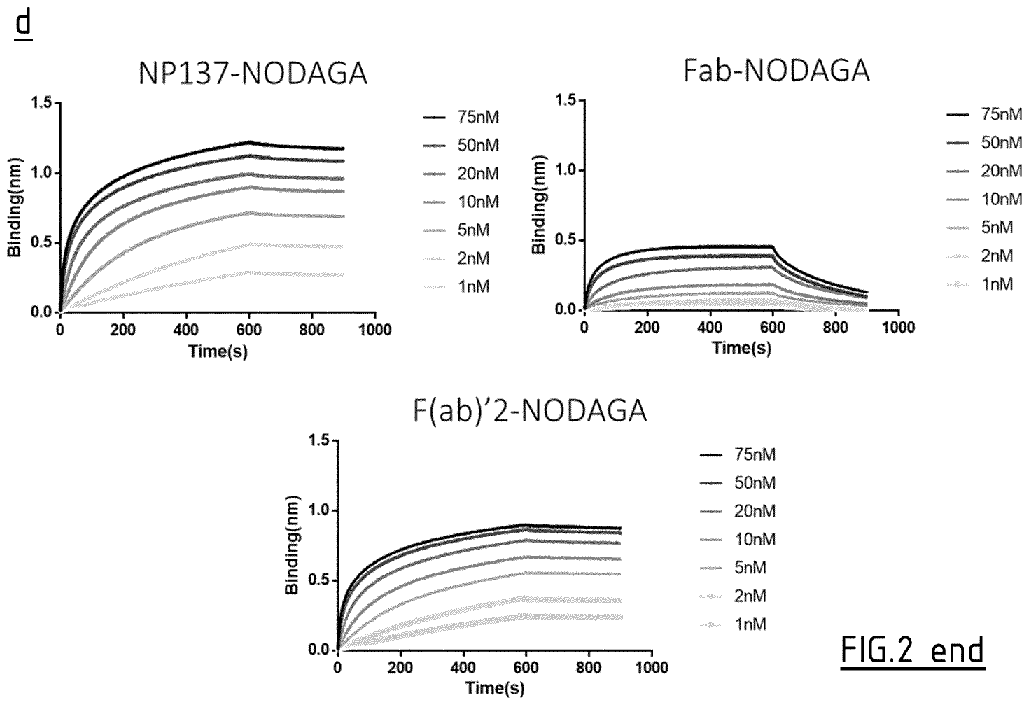

FIG. 2: Characterization of fragments conjugates. a. Chemical representation of DOTA (1,4,7,10-Tetraazacyclododecane-1,4,7,10-tetraacetic acid)-HS ester and NODAGA (1,4,7-Triazacyclononane, 1-glutaric acid-5,7 acetic acid)-HS ester molecule used for chelating metals b. Representation of full anti-Netrin-1 (NP137), F(ab)′2 and Fab conjugated to NODAGA or DOTA chelates. c. NP137), F(ab)′2 and Fab conjugates have been produced by synthesis and enzymatic cleavage and subjected to electrophoresis in denaturing or non-denaturing conditions. d. Bio-layer interferometry analysis of NP137), F(ab)′2 and Fab after chelating with NODAGA. Numbers indicates the concentration of NP137-NODAGA, F(ab)′2-NODAGA and Fab-NODAGA.

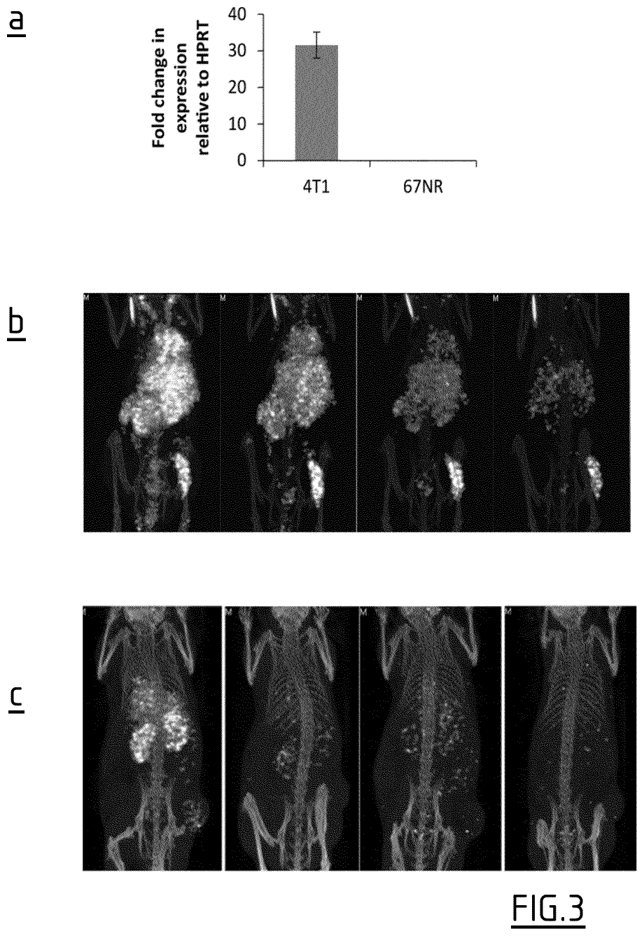

FIG. 3: SPECT/Ct analysis and netrin-1 detection in tumors. a. Quantification of netrin-1 expression by Q-RT-PCR on 4T1 and 67NR cell lines. b. Maximum Intensity Projection of Tomographic scintigraphy and X-ray CT of the whole body of a Balb/c mouse bearing a 4T1 tumor (positive for Netrin-1), acquired from the left to the right at 4h, 24h; 48h and 72h after IV injection of NP137-NODAGA-¹¹¹In. c. Maximum Intensity Projection of Tomographic scintigraphy and X-ray CT of the whole body of a Balb/c mouse bearing a 67NR (negative for Netrin-1) tumor, acquired from the left to the right at 24h; 48h and 72h after IV injection of NP137-NODAGA-¹¹¹In.

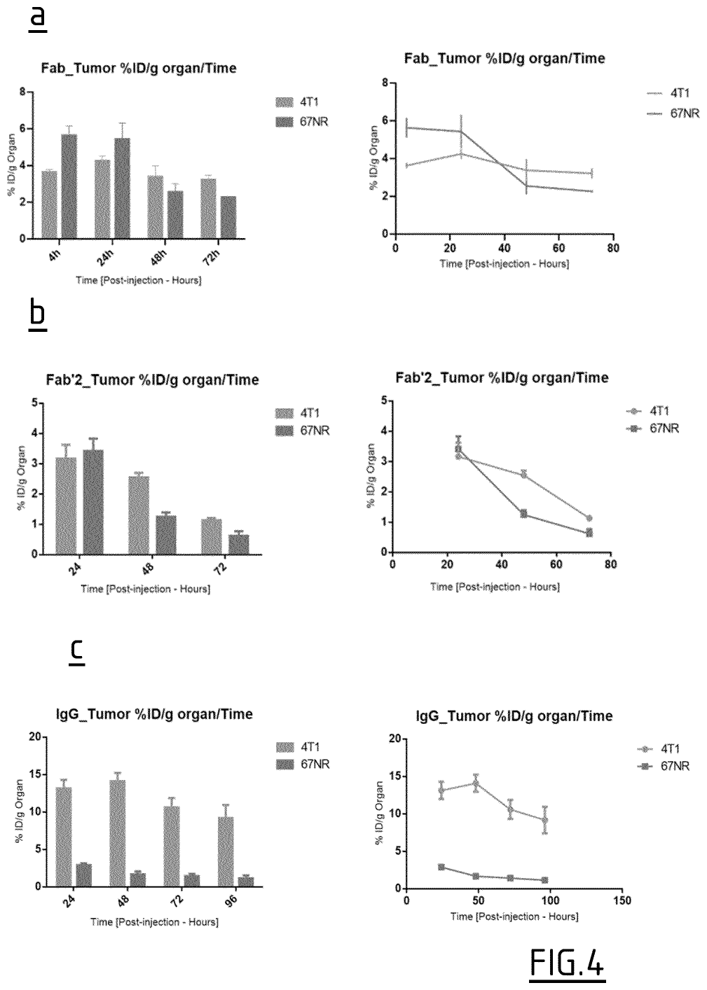

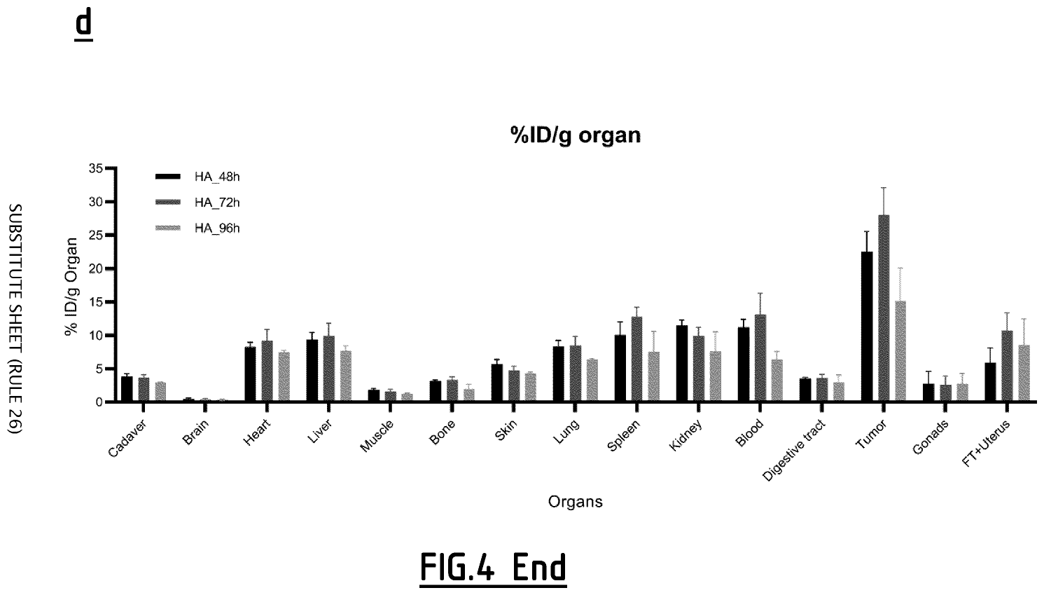

FIG. 4: Measurement of radioactivity accumulation. Tumoral biodistribution ratio of a. ¹¹¹In-NODAGA-NP137-Fab, b ¹¹¹In-NODAGANP137-F(ab)′₂ and c. -¹¹¹In-NODAGA-NP137 in Balb/cJ mouse bearing 4T1 xenografts (positive for netrin-1) versus 67NR xenografts (negative for netrin-1) at 24h, 48h, 72h and 96h. Radioactivity incorporation is quantified by the percentage of the injected dose by gramme of tumor. d. Biodistribution properties of ¹¹¹In-NODAGA-NP137 in Balb/cJ mouse bearing 4T1 xenografts at 48h, 72h and 96h and measured for all organs. Radioactivity incorporation is quantified by the percentage of the injected dose by gram of organ.

FIG. 5: Measurement of radioactivity accumulation

a. Maximum Intensity Projection of Tomographic scintigraphy and X-ray CT of the whole body of a MMTV/neuT mouse tumor genetically modified to develop mammary tumors, acquired from the left to the right at 24h; 48h and 72h after injection of NP137-NODAGA-¹¹¹In. b. Schematic representation and location of the 10 mammary glands in mice. c. Biodistribution properties of ¹¹¹In-NODAGA-NP137 in MMTV/NeuT mouse at 72h and measured for the tumors and all mice organs. Radioactivity incorporation is quantified by the percentage of the injected dose by gram of organ.

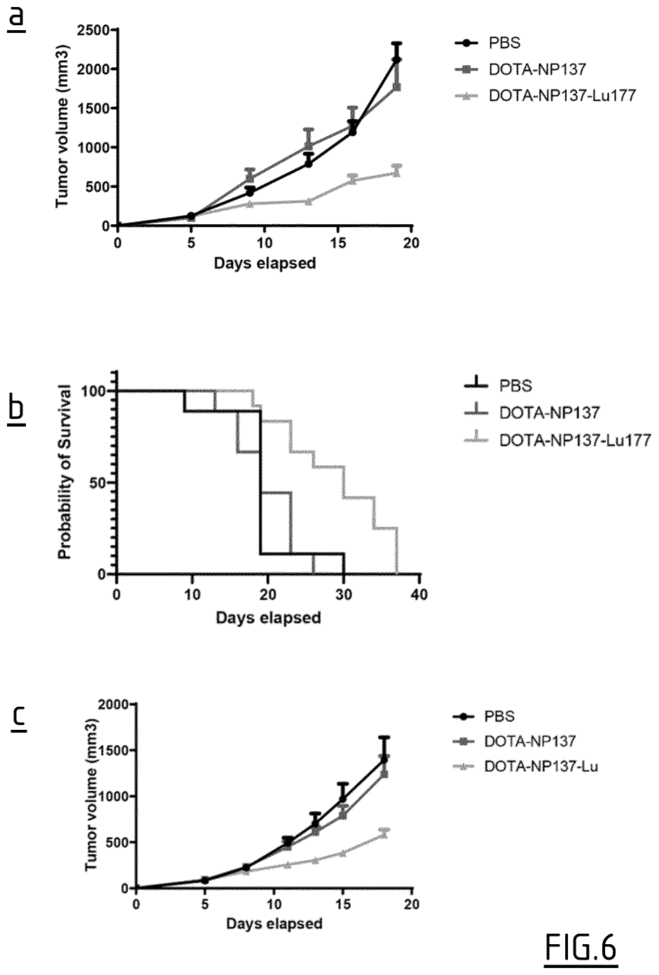

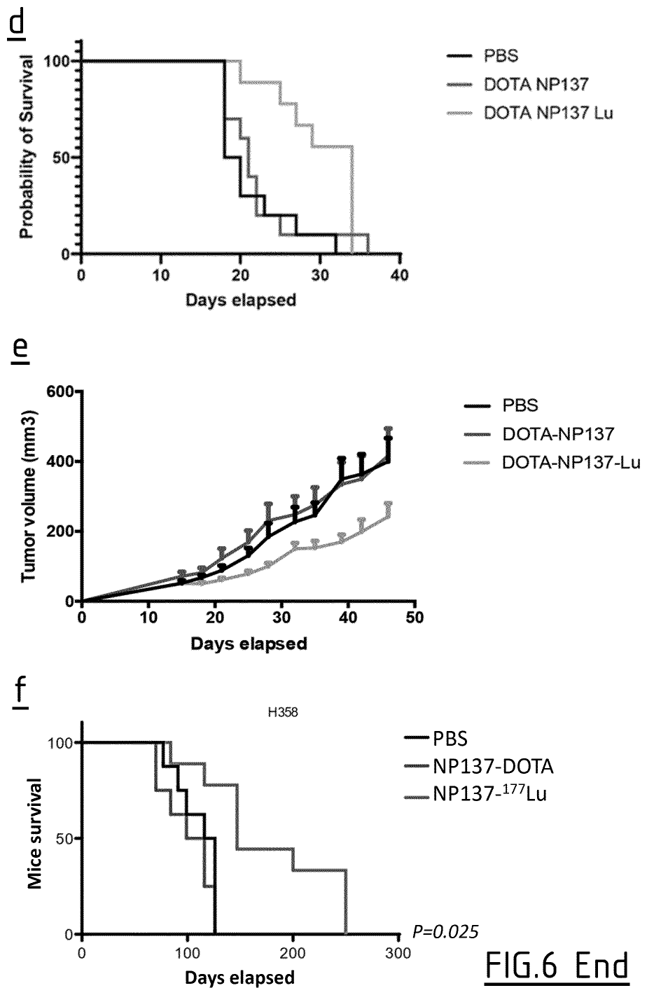

FIG. 6: A new anticancer therapy

a. Balb/cJ mice were engrafted with EMT6 cells by subcutaneous injection of 1 million cells. After 5 days, animals were treated by IV injection of PBS; DOTA-NP137 (anti-netrin1); DOTA-NP137-¹⁷⁷Lu. n=9 animals/group for PBS and DOTA-NP137; n=12 animals/group for DOTA-NP137-¹⁷⁷Lu; p<0.0001 between PBS and DOTA-NP137-¹⁷⁷Lu and between DOTA-NP137 and DOTA-NP137-¹⁷⁷Lu. b. DOTA-NP137-¹⁷⁷Lu enhances mice survival engrafted with EMT6 cell line (see A). Kaplan-Meier survival curves analysis of mice survival treated or not with NP137. Mantel Cox test; n=9 animals/group for PBS and DOTA-NP137; n=12 animals/group for DOTA-NP137-¹⁷⁷Lu; p<0.0001 between PBS and DOTA-NP137-¹⁷⁷Lu and between DOTA-NP137 and DOTA-NP137-¹⁷⁷Lu. c. Balb/c mice were engrafted with 4T1 cells by subcutaneous injection of 1 million cells. After 8 days, animals were treated by IV injection of PBS; DOTA-NP137 (anti-netrin-1); DOTA-NP137-¹⁷⁷Lu. n=5 animals/group for PBS and DOTA-NP137; n=6 animals/group for DOTA-NP137-¹⁷⁷Lu. d. DOTA-NP137-¹⁷⁷Lu enhances mice survival engrafted with 4T1 cell line (see c). Kaplan-Meier survival curves analysis of mice survival treated or not with NP137. Mantel Cox test; n=5 animals/group for PBS and DOTA-NP137; n=6 animals/group for DOTA-NP137-¹⁷⁷Lu; p<0.0001 between PBS and DOTA-NP137-¹⁷⁷Lu and between DOTA-NP137 and DOTA-NP137-¹⁷⁷Lu. e. NMRI nude mice were engrafted with SYO1 cells by subcutaneous injection of 5 million cells. After 8 days, animals were treated by IV injection of PBS; DOTA-NP137 (anti-netrin-1); DOTA-NP137-¹⁷⁷Lu. n=9 animals/group for PBS and DOTA-NP137; n=12 animals/group for DOTA-NP137-¹⁷⁷Lu; p<0.0001 between PBS and DOTA-NP137-¹⁷⁷Lu and between DOTA-NP137 and DOTA-NP137-¹⁷⁷Lu. f. NP137-¹⁷⁷Lu survival of mice engrafted with H358 cells. Kaplan-Meier survival curves of mice treated or not with DOTA-NP137. Mantel Cox test; n=8 animals/group for PBS and DOTA-NP137; n=9 animals/group for NP137-¹⁷⁷Lu; p=0.025 between PBS and NP137-¹⁷⁷Lu and between DOTA-NP137 and NP137-¹⁷⁷Lu.

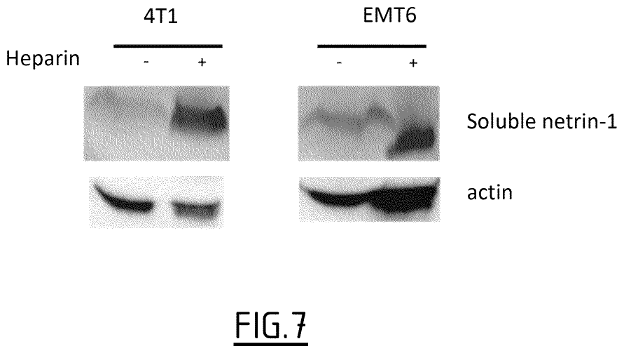

FIG. 7: Quantification of netrin-1 in concentrated supernatants from cells treated or not with heparin.

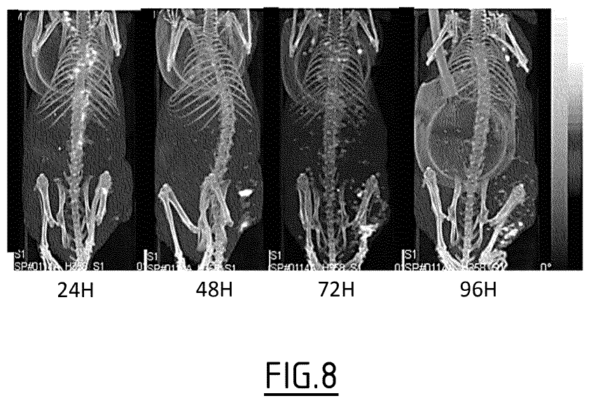

FIG. 8: Maximum Intensity Projection of Tomographic scintigraphy and X-ray CT of the whole body of a NMRI nude mouse bearing a H358 (netrin-1-positive) tumor, acquired at 24 h, 48 h, 72 h and 96 h after IV injection of NP137-NODAGA-111 In.

### MATERIALS AND METHODS

### Tumor Cell Lines

4T1 and 67NR murine mammary carcinoma cells were obtained from ATCC and cultured in RPMI-1640 (ATCC) medium supplemented with 10% foetal bovine serum (FBS, Gibco) and antibiotics (streptomycin and penicillin). EMT-6 murine mammary carcinoma cells were obtained from ATCC and cultured in Eagle Minimum Essential Medium (EMEM, ATCC) supplemented with 10% foetal bovine serum (FBS, Gibco) and antibiotics (streptomycin and penicillin). H358 human pulmonary adenocarcinoma H358 cells were obtained from ATCC and cultured in RPMI-1640 medium (ATCC) supplemented with 10% BBS (Gibco) and antibiotics. The cells were maintained in culture at 37° C. under a humidified atmosphere composed of 20% O₂ and 5% CO₂.

### Western Blots:

Confluent cells were washed with cold PBS and discarded in lysis buffer (Tris 10 mM pH 7.6; SDS 5; Glycerol 10%; Triton X-100 1%, DTT 100 mM). After sonication, the proteins were assayed using the Pierce 660 nm protein assay reagent (Thermo Fisher Scientific) and after loading onto SDS 4-15% polyacrylamide gels (Bio-Rad) transferred to nitrocellulose membranes using the Trans-Blot Turbo Transfer (Bio-Rad). The membranes were blocked for one hour at room temperature with 5% fat-free milk powder for Netrin-1 and with 5% BSA. Staining was performed overnight with a primary antibody: Netrin-1 antibody (Ab126729, Abcam). After washing, the membranes were incubated with a secondary antibody, an anti-goat rabbit antibody coupled with HRP for 1 hour at room temperature. The West Dura (Pierce) chemiluminescence system was used to intensify the signal. Imaging was performed using Chemidoch Touch (Bio-Rad).

For the binding of netrin-1 in cellular matrix, 1×10⁶ cells were plated in a 100 mm³ culture dish. 24 h after, cells were treated with 200 pg/mL of heparin sodium salt from porcine intestinal mucosa (H3147-100KU, Sigma) diluted in 4 mL of medium without FBS. After one night of incubation, supernatant was collected. Centricons centrifugal filters were used to concentrate the protein in the collected supernatant. Pierce 660 nm protein assay reagent (22660, Thermofisher scientific) was then used to determine the concentration of protein, 30 pg of proteins was loaded on immunoblots.

### In Vivo Preclinical Models:

The human monoclonal antibodies to NP137 (anti-netrin-1, HUM03) was kindly provided by Netris Pharma (Lyon, France). Female Balbc/J mice, 8-weeks old, were obtained from Janvier Laboratories (Le Genest-Saint-Isle, France). All syngeneic breast cancer cells 1×10⁶ EMT-6; 5×10⁵ 4T1 and 1×10⁶ 67-NR, were subcutaneously transplanted into the dorsal flank of 8-week-old female Balbc/J mice. The mice were maintained under specific pathogen-free conditions (Anican, Lyon—France and Imthernat facility, HCL Lyon, France) and stored in sterilized cages with filter lids. Their care and accommodation were in accordance with European and French institutional guidelines as defined by the local CECCAP Ethics Committee. The human cell lines H358 (1×10⁶ cells) or SKBR7 (2×10⁶ cells) were grafted onto 8 weeks female NMRI immunocompromised mice and maintained under the same conditions.

Tumor volume was assessed by measuring two perpendicular tumor diameters with a caliper three times a week. Individual tumor volumes were calculated as follows: V=(a*b2)/2. a being the largest diameter, b the smallest. When tumors reached a volume of 200-400 mm³, mice were randomly separated into groups of animals and subjected to treatment with either ¹¹¹In-NODAGA-NP137, ¹¹¹In-NODAGA-NP137-Fab, ¹¹¹In-NODAGA-NP137-F(ab′)₂ or ¹⁷⁷Lu-DOTA-NP137 and submitted to imagery/therapy. For all experiments, the mice were anaesthetized using a gas protocol (isoflurane/oxygen (2.5%/2.5%).

### Conjugation:

1 mL of the anti-Netrin1 monoclonal antibody-NP137 (or its fragments as appropriate, same conditions for all of them) is added on an Amicon Ultra-15 50 k (UFC905096). Diafiltration against 0.1 M phosphate buffer (pH 8) containing 1.2 g/L of Chelex 100 is performed. This step is repeated seven times using 10 mL of 0.1 M phosphate buffer (pH 8) solution with a 25 minutes centrifugation at 4900 rpm between each wash. Anti-Netrin 1 antibody concentration is then calculated with a nanodrop. The concentration of the antibody is then adjusted in order to be at 50 μM. Stock solution of DOTA-NHS ester/NODAGA (1,4,7-triazacyclononane, 1-glutaric acid-5,7 acetic acid)-HS ester (CheMatech (C084)) is dissolved in ultrapure water at a concentration of 10 mg/mL (=13.13 mM). 50 μM of anti-Netrin1 antibody is combined with required DOTA-NHS of NODAGA-NHS solution at a ratio of 1:25. Reactions are conducted at room temperature for 4h and transferred to 4° C. for continuous end-over-end mixing overnight. Diafiltration against PBS (Chelex) is performed. This step is repeated seven times using 10 mL of PBS (Chelex) with a 25 minutes centrifugation at 4900 rpm between each wash. DOTA/NODAGA-Anti-Netrin 1 antibody concentration is then calculated with a nanodrop.

### Radiolabeling:

NODAGA-NP137, NODAGA-NP137-Fab or NODAGA-NP137-F(ab′)₂ (40-70 μL, 5 mg/mL) were radiolabeled by adding 400 μL of 100 mM acetate buffer pH5 and 40-400 MBq of high purity ¹¹¹In-chloride (Covidien, Petten, The Netherlands). The mixture was incubated for 30 minutes at 37° C. The reaction was stopped with 100 μl of a 1 mM solution of DTPA. Free ¹¹¹In was removed using a PD-10 column. The column was first washed with 15 ml of 0.1 M acetate buffer, then the labelled mixture was loaded onto the column and eluted with the acetate buffer. ¹¹¹In-NODAGA-NP137, ¹¹¹In-NODAGA-NP137-Fab or ¹¹¹In-NODAGA-NP137-F(ab′)² were first eluted. The radiochemical purity (RCP) of each 0.5 ml fraction was evaluated using ITLC-SG (Biodex, Tec-control black) and 50 mM citrate buffer (pH5) as the mobile phase. Radiolabeled NP-137 remained at the origin while unbound ¹¹¹In migrated with an Rf of 0.9-1. The highest radiochemical purity fractions were pooled.

For stability testing, aliquots of radiolabeled ¹¹¹In-NODAGA-NP137, ¹¹¹In-NODAGA-NP137-Fab or ¹¹¹In-NODAGA-NP137-F(ab′)₂ were incubated at 37° C. in 2 mL phosphate buffered saline (pH 7.4) and the radiochemical purity (RCP) of the radiolabeled compounds was evaluated using ITLC-SG and 0.1 M citrate buffer pH5 as the mobile phase.

The same protocol may be applied for ¹¹¹In-DOTA-NP137, ¹¹¹In-DOTA-NP137-Fab or ¹¹¹In-DOTA-NP137-F(ab′)₂.

The same protocol was used to produce ¹⁷⁷Lu-DOTA-NP137, with DOTA-NHS.

### Biodistribution Studies

1 to 10 MBq of radiolabeled ¹¹¹In-NODAGA-NP137, ¹¹¹In-NODAGA-NP137-Fab or ¹¹¹In-NODAGA-NP137-F(ab′)₂ or ¹¹¹In DOTA-NP137 in a maximum volume of 100 μL were injected intravenously into tumour-bearing mice (n=3 or 4 for each group). The mice were sacrificed at defined times: 4h, 24h, 48h, 72h and 96h after injection by cervical dislocation. Tissues of interest (blood, heart, lungs, spleen, kidneys, muscles, brain and skin) were removed, weighted and the radioactivity was counted for 5 min in a gamma scintillation counter (Wizard® gamma counter, Perkin Elmer, USA). Urine and faeces were collected in an individual metabolic cage for housing and counting. Tissue distribution was expressed as a percentage of the injected dose per gram (% ID/g). Renal and hepatobiliary elimination was expressed as cumulative radioactivity under the total activity injected.

### Imaging:

The acquisitions were made using a Nano-SPECT/CT system for small animals (Bioscan, Washington, DC, USA). This system consists of four detectors (215×230 mm² NaI, 33 PMTs) equipped with interchangeable multipinhole openings. The SPECT/CT acquisitions were performed after IV injection of 5-15 MBq (mega Becquerel) of radiolabeled molecule at different times: 24h, 48h, 72h and 96h. CT (55 kVp tube voltage, 500 ms exposure time and 180 projections) and SPECT/CT acquisitions were performed in tumour-bearing mice in a supine position, placed in a temperature-controlled bed (Minerve, Esternay, France), in order to maintain body temperature (set at 37° C.). The acquisition was performed for 40 minutes with two 15% windows centered on the two peaks 171 keV and 245 keV of “¹′n. All image data were reconstructed and analyzed using the InVivo-Scope (Bioscan, Washington, DC, USA).

Generation of Fab and F(ab′)2 fragments and Synthesis of DOTA and NODAGA-Immunoconjugates

Proteolytic fragments of NP137 were generated using Pierce™ Fab and F(ab′)₂ Preparation kits according to manufacturer's instructions. For conjugation of DOTA or NODAGA to surface lysine residues, NP137 and its fragments were conjugated at a molar ratio of 25:1 chelate:antibody with DOTA-NHS-ester or NODAGA-NHS ester (Chematech, Dijon, France) in metal-free buffers prepared using Chelex 100 resin. Briefly, 50 μM antibodies were exchanged by diafiltration against 0.1 M phosphate buffer (pH8), then reacted with 1.25 mM DOTA-NHS-ester or NODAGA-NHS ester for 4h at 25° C. on a rotator. The reaction was transferred to 4° C. for continuous end-over-end mixing overnight. Excess chelator was removed by diafiltration against PBS. Immunoconjugates were stored at 4° C.

### Antibodies Affinity Determination

The affinity of the antibody fragments for netrin-1 were determined by biolayer interferometry using the OctetRed96 system (ForteBio) at 30° C. with constant shaking at 1000 rpm in PBS, 0.02% Tween-20, 0.1% BSA (BB). Briefly, recombinant human netrin-1 (R&D)-coated HIS1 K biosensors were incubated with a concentration series of antibody or fragments, and association was observed for 5 min. Biosensors were then incubated in BB for a further 5 min to observe dissociation of the complex. Binding kinetics were evaluated with ForteBio Octet RED Evaluation software 6.1 using a 1:1 binding model to derive kon, koff, and KD values.

### Results

### Netrin-1 is a Poorly Diffusible Matrix Binding Protein:

Immunohistochemistry (IHC) has been the reference for the characterization of target expression in cancer for years. However, this strategy has recently been called into question by the recent data obtained with immune checkpoint inhibitors, as there is a strong discrepancy between target expression and response within the patients. Thus, patients responding to the PDL-1 antibody could be negative for the expression of PDL-1 in IHC and vice versa. It can be hypothesized that target expression is not stable over time, and that IHCs are made with paraffin blocks taken with the diagnosis of the primary tumor and that target expression is different in metastases. New diagnostic strategies are therefore to be developed to analyze target expression in real time on a whole-body scale, to highlight all the variations in protein expression within tumors and metastasis.

The present inventors found that netrin-1 is not diffusible in tumour cells as was thought when describing the axon guidance growth model. They obtained netrin-1 immunohistochemistry pictures in endometrium and ovary human tumors paraffin embedded tumor sections (not shown). Netrin-1 in the human tumor was found to be present in the basement membrane of the cells after IHC staining, suggesting accumulation within the cell extracellular matrix. To complete and confirm this new finding, we have characterized molecular partners of Netrin-1 within the matrix components. A screen of interaction of netrin-1 with matrix proteins using bio-Layer interferometry (BLI) assays was realized. As a result, netrin-1 is able to strongly bind to Fibronectin, Laminin and Vitronectin (FIG. 1).

Heparin blocks interaction of netrin-1 to the plastic material. While no netrin-1 was detected in the conditioned medium from netrin-1-expressing 4T1/EMT6 cells in non-heparin treated condition, when heparin was added, netrin-1 was detected (FIG. 7).

All these elements imply that netrin-1 is sequestered in the extracellular matrix of cancer cells rather than diffusible.

### Characterization of a New Companion Test for Netrin-1 Real Time Detection:

Compounds were produced in which NP137 of fragments (Fab or F(ab′)2) were fused to Indium 111 (11 In) to be detected by SPECT/Ct molecular imaging (FIG. 2a). The three molecules formed are believed to have different molecular activities in vivo, the complete antibodies have a longer half-life in the bloodstream and the Fab's are able to penetrate more rapidly into the tumor, the F(ab′)₂ being an intermediate form (FIG. 2b). More precisely, the antibody or a fragment thereof has been conjugated to a metal chelator (DOTA or NODAGA) that binds to the lysine residues of the antibody or its fragment; and the chelator is associated or bound to the indium isotope, to complete the radiotracer. After steric exclusion chromatography purification, radiolabeled NP-137, Fab and F(ab′)₂ were obtained with radiochemical purity (RCP) exceeding 98%. Radiochemical yields were 70% for ¹¹¹In-NODAGA-NP137, 60% for ¹¹¹In-NODAGA-NP137-Fab and 65% for ¹¹¹In-NODAGA-NP137-F(ab′)₂. At 5 days after incubation, RCP was still greater than 95% in phosphate buffer saline (pH 7.4) indicating a suitable kinetic stability to perform in vitro and in vivo experiments. We show that this chemical modification, necessary to bind the isotope, did not disturb the ability of the three forms to bind Netrin-1 by bio-layer interferometry (FIG. 2d).

KD calculation after bio-layer interferometry analysis of the experiments presented in FIG. 2d show a high affinity KD as follows:
NP137-NODAGA: 1.72 E-10
F(ab)′₂-NODAGA: 1.51 E-10
Fab-NODAGA: 1.52 E-10.

To analyze the capacity of these molecules to detect Netrin-1 in vivo, we used two syngeneic models of tumor: 4T1 cells positive for Netrin-1 expression and 67NR negative for Netrin-1 as a negative control.

First, quantification of netrin-1 expression by Q-RT-PCR on 4T1 and 67NR cell lines was performed. Results on FIG. 3a confirm that there is netrin-1 expression only in the 4T1 cells.

Second, Maximum Intensity Projection of Tomographic scintigraphy and X-ray CT of the whole body of a Balb/cJ mice bearing a 4T1(positive for Netrin-1) tumor, acquired at 24h; 48h; 72h and 96h after IV injection of ¹¹¹In-NODAGA-NP137-F(ab)′₂, ¹¹¹In-NODAGA-NP137-Fab, or ¹¹¹In-NODAGA-NP137 was obtained. Similarly, Maximum Intensity Projection of Tomographic scintigraphy and X-ray CT of the whole body of a Balb/c mouse bearing a 67NR (negative for Netrin-1) tumor, acquired at 24h; 48h and 72h after IV injection of ¹¹¹In-NODAGA-NP137-F(ab)′₂, ¹¹¹In-NODAGA-NP137-Fab, or ¹¹¹In-NODAGA-NP137 was obtained.

A strong tumor intake is detectable in 4T1 tumors in ¹¹¹In-NODAGA-NP137 group (FIG. 3b), whereas slower intake was detectable in ¹¹¹In-NODAGA-NP137-F(ab)′₂ group and in ¹¹¹In-NODAGA-NP137-Fab group (data not shown). Interestingly no intake could be detected for all molecules in 67NR bearing mice, implicating that tumoral intake is specific of tumoral Netrin-1 (compare FIGS. 3B and 3C). Localized tumor intake is also visible in H358 tumor at FIG. 8.

We performed ex vivo quantification of the intake ratio between 67NR and 4T1 cells implicating that the best specific incorporation is detected 48h after treatment (FIG. 4a-c). These data confirmed that tumor incorporation is better with full antibody than other forms.

Biodistribution properties of ¹¹¹In-NODAGA-NP137 in Balb/cJ mouse bearing 4T1 xenografts at 48h, 72h and 96h was measured for all organs. Radioactivity incorporation is quantified by the percentage of the injected dose by gram of organ. A strong tumor intake close to 25% of the injected dose (ID) was detected in the tumor after the indicated time laps. The incorporation within other organs did not reveal a specific bindings (FIG. 4d).

### A New Diagnostic Tool

A further test was done in a transgenic model of mammary luminal breast cancer MMTV-NeuT (20), expressing endogenous levels netrin-1. FIG. 5a shows the evolution in time, from the left to the right at 24h, 48h and 72h after injection of ¹¹¹In-NODAGA-NP137.

A strong staining was detected in fat pad tissue, and this in the 10 mammary glands of the animals (see FIG. 5b).

It is very interesting to note that some tumors were visualized even before being detected by mammary palpation, which reinforces the relevance of this tracer as an early detecting tool for netrin-1 expressing tumors. This result was unexpected. A strong tumor intake close to 8% of the injected dose (ID) could be detectable after a tumor incorporation measurement, in all tumors arguing that “¹¹¹In NODAGA-NP137 could be a good diagnosis tool to describe the expression of Netrin-1 upon the appearance of a small lesion in term of tumor mass. (FIG. 5c).

FIG. 8 show the strong accumulation of NP137-NODAGA-¹¹¹In in the netrin-1-positive human xenograft murine model H358 (human non-small cell lung cancer model). Similar results (not shown) were obtained with EMT6 (murine mammary carcinoma cell line. Tumoral uptakes in both models were more than 10% of ID/g (injected dose/weigh of organ in gram).

NP137-DOTA-¹⁷⁷Lu a new theranostic compound to target resistant tumors.

We have developed an antibody fused to Lutecium 177 (called DOTA-NP137-¹⁷⁷Lu on FIG. 6) that will emit beta radiation that effectively damages cancer cells. Lutecium will emit a strong dose of radiation in a range of 1.8 mm, with high specificity when coupled to a targeted therapy.

We first use this molecule to treat the 4T1 and the EMT6 cell lines. These cell lines are resistant to NP137 as a single agent and are known to belongs to the most aggressive preclinical models. Nevertheless, tumor growth decreased when treated with a single 10MBq dose of NP137-DOTA-¹⁷⁷Lu compare to control groups with either PBS or DOTA-NP137 (FIGS. 6a, c and e). As a consequence, survival of mice was increased in the NP137-DOTA-¹⁷⁷Lu treated group (FIGS. 6b and d). These results indicates that this new molecule could lead to antitumoral activities in vivo in tumor expressing netrin-1.

Therapeutic efficacy of NP137-¹⁷⁷Lu was further assessed in the human lung cancer xenograft model H358 where the treatment again significantly reduced the rate of tumor growth corroborating a clear anti-tumoral effect (p<0.001) (FIG. 6f).

### Discussion

In this study, we described a new companion test for the in vivo detection of netrin-1 by nuclear medicine SPECT/CT. Netrin-1 has been characterized as a therapeutic target in several types of cancer currently evaluated in clinical assays, but due to the lack of assay in a conventional test (i.e., serum detection with Elisa assays, mass spectrometry, robustness of pathology revealing netrin-1 in FFPE samples), we developed an innovative, simple and robust companion test to detect the high expression of netrin-1 in vivo in cancer cells. Based on our results, we can state that no or very-low a specific binding of ¹¹¹In-NODAGA-NP137 or ¹¹¹In-DOTA-NP137 detected in our preclinical models, as revealed by the RCP in the netrin-1 negative tumor model 67NR. These results show that netrin-1 is not widely expressed at the adult level, and show a high specificity for tumoral tissues. The targeting of the developmental Netrin-1 genes that are re-expressed during tumor formation therefore appears to be a key solution for improving the specificity of tumor imaging.

To identify the best molecule for SPECT or PET imaging, we designed three different agents based on the clinically used human anti-netrin-1 NP137 monoclonal antibody: NP137-IgG1 complete, NP137-F(ab′)2 and NP137-Fab. All of these molecules can bind strongly to netrin-1. The best accumulation in vivo with tumour specificity was observed for the radiotracer containing full NP137-IgG1 and NP137-Fab; the best was the radiotracer containing the full antibody. Complete NP137-IgG1 showed the best accumulation within the tumour and the most promising results for transfer within the clinic. This transfer could be hypothesized with all the metals and compounds used for molecular imaging. In terms of more basic research, Netrin-1 has been described for years in neural development as a secreted molecule with a diffusible gradient. Netrin-1 is a ligand and as such is not a primary choice as the target of imagery or internal radiotherapy.

Moreover, and based on this demonstrated tumor incorporation we design a new molecule in which we fused NP137-DOTA with Lutecium 177 to form NP137-DOTA-¹⁷⁷Lu. As a result, this molecule also accumulated specifically within the tumors expressing netrin-1. Lutecium 177 is a B-emitter able to deliver a strong dose of radiation in a range of 1.8 mm within the tumoral tissue. As consequence, we note a significant decrease in tumor growth correlated with a better survival on mice bearing tumor. It is remarkable to note that these tumor types were totally resistant to NP137 as a single agent. The survival studies demonstrate that treatment with this compound increased mice life two times with respect to control, in mice tumor models and in human tumor model. As NP137 has proven its safety as a single agent and doses ¹⁷⁷Lu are well characterized, so that this molecule can transferred to treat tumors in a simple manner.

1.-17. (canceled)

18. A compound comprising:
an anti-Netrin-1 antibody or antigen binding fragment thereof, and
a chelating moiety bound to said antibody or fragment.

19. The compound of claim 18, wherein said chelating moiety is associated with a radioisotope.

20. The compound of claim 18, wherein the antibody is a monoclonal antibody or an antigen-binding fragment thereof, comprising
a variable domain VH comprising:
a H-CDR1 having a sequence set forth as SEQ ID NO: 1;
a H-CDR2 having a sequence set forth as SEQ ID NO: 2;
a H-CDR3 having a sequence set forth as SEQ ID NO: 3;
a variable domain VL comprising:
a L-CDR1 having a sequence set forth as SEQ ID NO: 4;
a L-CDR2 having a sequence YAS;
a L-CDR3 having a sequence set forth as SEQ ID NO: 5;
or
a variable domain VH comprising:
a H-CDR1 having a sequence set forth as SEQ ID NO: 22;
a H-CDR2 having a sequence set forth as SEQ ID NO: 23;
a H-CDR3 having a sequence set forth as SEQ ID NO: 24;
a variable domain VL comprising:
a L-CDR1 having a sequence set forth as SEQ ID NO: 25;
a L-CDR2 having a sequence set forth as SEQ ID NO: 26;
a L-CDR3 having a sequence set forth as SEQ ID NO: 5.

21. The compound of claim 18, wherein the antibody is a monoclonal antibody or an antigen-binding fragment thereof, wherein the antibody is a monoclonal antibody or an antigen-binding fragment thereof, comprising a pair of VH and VL sequences selected from the following pairs: SEQ ID NO: 21 and 13, SEQ ID NO: 14 and 8, SEQ ID NO: 15 and 9, SEQ ID NO: 16 and 10, SEQ ID NO: 17 and 11, SEQ ID NO: 18 and 11, SEQ ID NO: 19 and 10, SEQ ID NO: 20 and 11, SEQ ID NO: 16 and 11, SEQ ID NO: 19 and 12, SEQ ID NO: 15 and 10.

22. The compound of claim 21, wherein the antibody further comprises a Human IgG1 Constant heavy chain (CH) and/or a Human IgG1 Constant light chain (CL).

23. The compound of claim 18, wherein the chelating moiety comprises NODAGA, NODAGA-NHS, DOTA, DOTA-NHS, p-SCN-Bn-NOTA, p-SCN-Bn-PCTA, p-SCN-Bn-oxo-DO3A, desferrioxamine-p-SCN, Diethylenetriamine Pentaacetic Acid (DTPA), or 1,4,8,11-Tetraazacyclotetradecane-1,4,8,11-tetraacetic acid (TETA).

24. The compound of claim 19, wherein the radioisotope is ⁶⁸Ga, ⁶⁴Cu, ⁸⁹Zr, ¹⁸⁶Re, ¹⁸⁸Re, ¹⁵³Sm, ¹¹¹In, ^{99m}Tc, ¹²³I, ¹⁷⁷Lu, ⁹⁰Y, ¹³¹I, ²¹³Bi, ²¹²Bi, ²¹¹At or ²²⁵Ac.

25. A method for imaging Netrin-1 presence or localization in a subject, comprising:
a) administering to said subject a compound comprising:
an anti-Netrin-1 antibody or antigen binding fragment thereof,
a chelating moiety bound to said antibody or fragment, and
a radioisotope associated to the chelating moiety,
b) waiting from 4 to 172 h, obtaining binding of said compound by the netrin-1 sequestered in the extracellular matrix of the tumor;
c) detecting or localizing said bound compound by in vivo imaging.

26. The method of claim 25, wherein localizing at step b) comprises highlighting the presence or accumulation of the compound in at least one body part.

27. The method of claim 26, wherein said body part is an organ or a tissue.

28. The method of claim 25, wherein the radioisotope is selected from the group consisting of ⁶⁸Ga, ⁶⁴Cu, ⁸⁹Zr, ¹⁸⁶Re, ¹⁸⁸Re, ¹⁵³Sm, ¹¹¹In, ^{99m}Tc, and ¹²³I.

29. A method of internal radiotherapy treatment of a netrin-1 expressing cancer in a patient having such cancer, comprising administering a sufficient amount of a compound comprising:
an anti-Netrin-1 antibody or antigen binding fragment thereof,
a chelating moiety bound to said antibody or fragment, and
a radioisotope associated to the chelating moiety.

30. The method of claim 29, wherein the compound comprises a radioisotope selected from the group consisting of ¹⁷⁷Lu, ⁹⁰Y, ¹³¹I, ²¹³Bi, ²¹²Bi, ²¹¹At and ²²⁵Ac.

31. A method of identifying and treating patients with a netrin-1 expressing cancer, comprising:
a) administering to said subject a compound comprising:
an anti-Netrin-1 antibody or antigen binding fragment thereof,
a chelating moiety bound to said antibody or fragment, and
a radioisotope associated to the chelating moiety;
b) waiting from 4 to 172 h, obtaining binding of said compound by the netrin-1 sequestered in the extracellular matrix of the tumor;
c) detecting said compound by in vivo imaging;
d) visualizing localization of Netrin-1 presence or accumulation;
e) treating said patient against the visualized cancer.

32. The method of claim 31 wherein the compound in a) comprises a radioisotope selected from the group consisting of ⁶⁸Ga, ⁶⁴Cu, ⁸⁹Zr, ¹⁸⁶Re, ¹⁸⁸Re, ¹⁵³Sm, ¹¹¹In, ^{99m}Tc, and ¹²³I.

33. The method of claim 31, wherein treating the patient in e) comprises administering to said patient an effective amount of a compound comprising:
an anti-Netrin-1 antibody or antigen binding fragment thereof,
a chelating moiety bound to said antibody or fragment, and
a radioisotope associated to the chelating, wherein the radioisotope is selected from the group consisting of 1⁷⁷Lu, ⁹⁰Y, ¹³¹I, ²¹³Bi, ²¹²Bi, ²¹¹At and ²²⁵Ac.

34. The method of claim 31, wherein treating the patient in e) comprises administering to said patient an effective amount of a monoclonal antibody or an antigen-binding fragment thereof, comprising
a variable domain VH comprising:
a H-CDR1 having a sequence set forth as SEQ ID NO: 1;
a H-CDR2 having a sequence set forth as SEQ ID NO: 2;
a H-CDR3 having a sequence set forth as SEQ ID NO: 3;
a variable domain VL comprising:
a L-CDR1 having a sequence set forth as SEQ ID NO: 4;
a L-CDR2 having a sequence YAS;
a L-CDR3 having a sequence set forth as SEQ ID NO: 5;
or
a variable domain VH comprising:
a H-CDR1 having a sequence set forth as SEQ ID NO: 22;
a H-CDR2 having a sequence set forth as SEQ ID NO: 23;
a H-CDR3 having a sequence set forth as SEQ ID NO: 24;
a variable domain VL comprising:
a L-CDR1 having a sequence set forth as SEQ ID NO: 25;
a L-CDR2 having a sequence set forth as SEQ ID NO: 26;
a L-CDR3 having a sequence set forth as SEQ ID NO: 5.

35. The method of claim 34, wherein the antibody is a monoclonal antibody or an antigen-binding fragment thereof, wherein the antibody is a monoclonal antibody or an antigen-binding fragment thereof, comprising a pair of VH and VL sequences selected from the following pairs: SEQ ID NO: 21 and 13, SEQ ID NO: 14 and 8, SEQ ID NO: 15 and 9, SEQ ID NO: 16 and 10, SEQ ID NO: 17 and 11, SEQ ID NO: 18 and 11, SEQ ID NO: 19 and 10, SEQ ID NO: 20 and 11, SEQ ID NO: 16 and 11, SEQ ID NO: 19 and 12, SEQ ID NO: 15 and 10.

36. The method of claim 31, wherein in vivo imaging comprises PET or SPECT imaging.

37. A compound comprising:
an anti-Netrin-1 antibody or antigen binding fragment thereof, and
a chelating moiety bound to said antibody or fragment.
wherein said chelating moiety is optionally associated with a radioisotope and wherein the antibody is a monoclonal antibody or an antigen-binding fragment thereof, comprising
a variable domain VH comprising:
a H-CDR1 having a sequence set forth as SEQ ID NO: 1;
a H-CDR2 having a sequence set forth as SEQ ID NO: 2;
a H-CDR3 having a sequence set forth as SEQ ID NO: 3;
a variable domain VL comprising:
a L-CDR1 having a sequence set forth as SEQ ID NO: 4;
a L-CDR2 having a sequence YAS;
a L-CDR3 having a sequence set forth as SEQ ID NO: 5;
or
a variable domain VH comprising:
a H-CDR1 having a sequence set forth as SEQ ID NO: 22;
a H-CDR2 having a sequence set forth as SEQ ID NO: 23;
a H-CDR3 having a sequence set forth as SEQ ID NO: 24;
a variable domain VL comprising:
a L-CDR1 having a sequence set forth as SEQ ID NO: 25;
a L-CDR2 having a sequence set forth as SEQ ID NO: 26;
a L-CDR3 having a sequence set forth as SEQ ID NO: 5.
